(19) United States
(12) Patent Application Publication
(10) Pub. No.: US 2016/0319253 A1
(43) Pub. Date: Nov. 3, 2016

(54) NOVEL FUSION GENES AS FACTORS RESPONSIBLE FOR GASTRIC CANCER

(71) Applicants: NATIONAL CANCER CENTER, Tokyo (JP); LSIP, LLC, Tokyo (JP)

(72) Inventors: Tatsuhiro SHIBATA, Tokyo (JP); Fumie HOSODA, Tokyo (JP)

(21) Appl. No.: 15/105,141

(22) PCT Filed: Dec. 18, 2014
(86) PCT No.: PCT/JP2014/083526

§ 371 Date: Jun. 16, 2016

(30) Foreign Application Priority Data
Dec. 19, 2013 (JP) 2013-263032

(51) Int. Cl. C12N 9/12; C07K 14/475; C12N 9/00; C07K 14/705; C12Q 1/68; C12N 15/113

(52) U.S. Cl. CPC C12N 9/12; C12Q 1/6886; C12N 15/113; C12N 15/1137; C12N 9/93; C12Y 603/02; C12Y 207/10001; C12Y 207/11001; C07K 14/705; C07K 14/4756; C12Q 2600/156; C12Q 2600/136; C12N 2310/14; C07K 2319/00

(57) ABSTRACT

To identify mutations that can serve as indicators for predicting the effectiveness of drug treatments in cancers such as gastric cancer; to provide a means for detecting said mutations; and to provide a means for identifying, based on said mutations, patients with cancer or subjects with a risk of cancer, in whom drugs targeting genes having said mutations or proteins encoded by said genes show a therapeutic effect.

A method for detecting a gene fusion serving as a responsible mutation (driver mutation) for cancer, the method comprising the step of detecting any one of an ATG3-EPHB1 fusion polynucleotide, a TNIK-RNF123 fusion polynucleotide, or an SLC12A2-NRG2 fusion polynucleotide, or a polypeptide encoded thereby, in an isolated sample from a subject.

### TECHNICAL FIELD

The present invention relates mainly to a method for detecting gene fusions serving as responsible mutations for cancer, and a method for identifying patients with cancer or subjects with a risk of cancer, in whom substances suppressing the expression and/or activity of polypeptides encoded by fusion polynucleotides produced by said gene fusions show a therapeutic effect.

### BACKGROUND ART

Cancer is the first-ranked disease among causes of death in Japan. Cancer cells are known to become malignant due to genetic alterations. Identifying genetic alterations characteristic of cancer type is expected to contribute not only to the clarification of carcinogenic mechanisms but also to, for example, the diagnosis and prognosis prediction of cancers and the development and selection of a cancer therapy.

There have hitherto been reported many cancer-related genes.

For example, EPHB1 is a gene encoding a receptor tyrosine kinase involved in the development of the nervous system and other tissues. It was reported that loss or reduction of the expression of this protein is associated with invasion and metastasis in gastric cancers (Non Patent Literature 1), invasion in colorectal cancers (Non Patent Literature 2), and the like.

Also, it was reported that the TNIK (Traf2- and Nck-interacting kinase) gene is essential for colorectal cancer growth (Non Patent Literature 3) and may have an impact on breast cancer cell growth (Non Patent Literature 4).

The SLC12A2 gene, also called NKCC1 (Na—K—Cl cotransporter 1), was suggested to modulate the migration of glioma cells (Non Patent Literature 5).

NRG2 is a gene encoding a protein in the neuregulin family which controls cell growth and differentiation through interaction with ERBB family receptors. It was shown that high expression of this gene is related to the aggressiveness of tumors (Non Patent Literature 6).

In recent years, molecular targeted drugs against cancers have been developed, and there have been increasing examples of successful development of molecular targeted therapies against constituents (e.g., kinases) of carcinogenesis-related signaling pathways. For example, it is reported that tyrosine kinase inhibitors (e.g., crizotinib) targeting ALK protein are effective in the treatment of lung cancer patients bearing the EML4-ALK fusion gene, and that tyrosine kinase inhibitors (e.g., imatinib) targeting the protein encoded by the BCR-ABL fusion gene are effective in the treatment of leukemia patients bearing this fusion gene.

According to a certain international comparison of morbidity rate, gastric cancer is known to be highly prevalent in East Asian countries including Japan as well as in South America. A comparison of cancer deaths in Japan by body part shows that gastric cancer is the second most frequent cause of cancer deaths in both men and women. In molecular therapies against gastric cancer, there are several known gene targets including HER2.

### CITATION LIST

### Non Patent Literatures

[Non Patent Literature 1] Wang J. D., et al., Oncology, 2007, 73 (3-4), 238-45.
[Non Patent Literature 2] Sheng Z., et al., Pathobiology, 2008, 75 (5), 274-80.
[Non Patent Literature 3] shitashige M., et al., Cancer Res, 2010, 70 (12), 5024-33.
[Non Patent Literature 4] Jiao X., et al., BMC Genomics, 2013, 14, 165.
[Non Patent Literature 5] Garzon-Muvdi T., et al., PLoS Biol, 2012, 10 (5), e1001320.
[Non Patent Literature 6] Revillion F., et al., Ann Oncol, 2008, 19 (1), 73-80.

### SUMMARY OF INVENTION

### Technical Problem

Identification of mutations in various cancers including gastric cancer has not yet been made thoroughly, and there is still a demand for further identification of mutations that can serve as therapeutic targets and as indicators for predicting the effectiveness of drug treatments.

Accordingly, the objects of the present invention include but are not limited to the following: to identify mutations that can serve as an indicator for predicting the effectiveness of drug treatments in various cancers including gastric cancer; to provide a means for detecting said mutations; and to provide a means for identifying, based on said mutations, patients with cancers or subjects with a risk of cancers, in whom drugs targeting genes having said mutations or proteins encoded by said genes show a therapeutic effect.

### Solution to Problem

As the result of conducting intensive studies with a view to achieving the above-mentioned objects, the present inventors identified kinase fusion genes (ATG3-EPHB1 and TNIK-RNF123) and a kinase-related molecule fusion gene (SLC12A2-NRG2) as novel genomic aberrations in gastric cancer. None of these fusion genes is expressed in normal gastric mucosal tissues, and besides, none of the known causative gene mutations for gastric cancer (i.e., KRAS, NRAS and BRAF mutations) was detected in case patients in whom any of the above-mentioned fusion genes was detected; thus, these fusion genes were considered as responsible mutations (driver mutations) for cancer.

The ATG3-EPHB1 and TNIK-RNF123 fusion genes were considered to lead to constitutive activation of EPHB1 and TNIK kinases, respectively, thereby contributing to malignant transformation of cancers. Therefore, it is considered that EPHB1 and TNIK kinase inhibitors, respectively, would produce a therapeutic effect on cancers positive for the above-mentioned gene fusions.

As regards the SLC12A2-NRG2 fusion gene, since the cell growth factor NRG2 is a ligand for HER2 which has already been known as a therapeutic target for gastric cancer, it was believed that this fusion gene contributes to the development of gastric cancer through a similar molecular mechanism to that of HER2 mutation. Therefore, an antibody drug against NRG2, or antibody drugs or kinase inhibitors against a group of HER proteins serving as NRG2 receptors could produce a therapeutic effect on cancer positive for this gene fusion.

The present inventors have made further studies based on these findings and completed the present invention.

More specifically, the present invention is defined as follows.

[1] A method for detecting a gene fusion serving as a responsible mutation (driver mutation) for cancer, the method comprising the step of detecting a fusion polynucleotide of any one of (a) to (c) below, or a polypeptide encoded thereby, in an isolated sample from a subject:
(a) an ATG3-EPHB1 fusion polynucleotide which encodes a polypeptide comprising the kinase domain of EPHB1 and having kinase activity;
(b) a TNIK-RNF123 fusion polynucleotide which encodes a polypeptide comprising the kinase domain of TNIK and the SPRY domain and RING finger domain of RNF123, and having kinase activity; and
(c) an SLC12A2-NRG2 fusion polynucleotide which encodes a polypeptide comprising the AA_permease_N super family domain of SLC12A2 and the neuregulin family conserved region of NRG2, and having intracellular signaling-enhancing activity.
[2] The method according to [1], wherein the fusion polynucleotide is any one of (a) to (c) below:
(a) an ATG3-EPHB1 fusion polynucleotide encoding the polypeptide mentioned below:

(i) a polypeptide consisting of the amino acid sequence of SEQ ID NO: 2,

(ii) a polypeptide consisting of an amino acid sequence derived from the amino acid sequence of SEQ ID NO: 2 by deletion, substitution or addition of one or more amino acids, and the polypeptide having kinase activity, or

(iii) a polypeptide consisting of an amino acid sequence having a sequence identity of at least 80% to the amino acid sequence of SEQ ID NO: 2, and the polypeptide having kinase activity;

(b) a TNIK-RNF123 fusion polynucleotide encoding the polypeptide mentioned below:

(i) a polypeptide consisting of the amino acid sequence of SEQ ID NO: 4, 6, 8, 10, 12, 14, 16 or 18,

(ii) a polypeptide consisting of an amino acid sequence derived from the amino acid sequence of SEQ ID NO: 4, 6, 8, 10, 12, 14, 16 or 18 by deletion, substitution or addition of one or more amino acids, and the polypeptide having kinase activity, or

(iii) a polypeptide consisting of an amino acid sequence having a sequence identity of at least 80% to the amino acid sequence of SEQ ID NO: 4, 6, 8, 10, 12, 14, 16 or 18, and the polypeptide having kinase activity; and

(c) an SLC12A2-NRG2 fusion polynucleotide encoding the polypeptide mentioned below:

(i) a polypeptide consisting of the amino acid sequence of SEQ ID NO: 20, 22, 24, 26 or 28,

(ii) a polypeptide consisting of an amino acid sequence derived from the amino acid sequence of SEQ ID NO: 20, 22, 24, 26 or 28 by deletion, substitution or addition of one or more amino acids, and the polypeptide having intracellular signaling-enhancing activity, or

(iii) a polypeptide consisting of an amino acid sequence having a sequence identity of at least 80% to the amino acid sequence of SEQ ID NO: 20, 22, 24, 26 or 28, and the polypeptide having intracellular signaling-enhancing activity.

[3] The method according to [1] or [2], wherein the fusion polynucleotide is any one of (a) to (c) below:
(a) (i) a polynucleotide consisting of the nucleotide sequence of SEQ ID NO: 1,

(ii) a polynucleotide that hybridizes under stringent conditions with a polynucleotide consisting of a nucleotide sequence complementary to the polynucleotide consisting of the nucleotide sequence of SEQ ID NO: 1, and which encodes a polypeptide having kinase activity,

(iii) a polynucleotide that consists of a nucleotide sequence derived from the nucleotide sequence of SEQ ID NO: 1 by deletion, substitution or addition of one or more nucleotides, and which encodes a polypeptide having kinase activity, or

(iv) a polynucleotide that has a sequence identity of at least 80% to the polynucleotide consisting of the nucleotide sequence of SEQ ID NO: 1, and which encodes a polypeptide having kinase activity;

(b) (i) a polynucleotide consisting of the nucleotide sequence of SEQ ID NO: 3, 5, 7, 9, 11, 13, 15 or 17,

(ii) a polynucleotide that hybridizes under stringent conditions with a polynucleotide consisting of a nucleotide sequence complementary to the polynucleotide consisting of the nucleotide sequence of SEQ ID NO: 3, 5, 7, 9, 11, 13, 15 or 17, and which encodes a polypeptide having kinase activity,

(iii) a polynucleotide that consists of a nucleotide sequence derived from the nucleotide sequence of SEQ ID NO: 3, 5, 7, 9, 11, 13, 15 or 17 by deletion, substitution or addition of one or more nucleotides, and which encodes a polypeptide having kinase activity, or

(iv) a polynucleotide that has a sequence identity of at least 80% to the polynucleotide consisting of the nucleotide sequence of SEQ ID NO: 3, 5, 7, 9, 11, 13, 15 or 17, and which encodes a polypeptide having kinase activity; and

(c) (i) a polynucleotide consisting of the nucleotide sequence of SEQ ID NO: 19, 21, 23, 25 or 27,

(ii) a polynucleotide that hybridizes under stringent conditions with a polynucleotide consisting of a nucleotide sequence complementary to the polynucleotide consisting of the nucleotide sequence of SEQ ID NO: 19, 21, 23, 25 or 27, and which encodes a polypeptide having intracellular signaling-enhancing activity,

(iii) a polynucleotide that consists of a nucleotide sequence derived from the nucleotide sequence of SEQ ID NO: 19, 21, 23, 25 or 27 by deletion, substitution or addition of one or more nucleotides, and which encodes a polypeptide having intracellular signaling-enhancing activity, or

(iv) a polynucleotide that has a sequence identity of at least 80% to the polynucleotide consisting of the nucleotide sequence of SEQ ID NO: 19, 21, 23, 25 or 27, and which encodes a polypeptide having intracellular signaling-enhancing activity.

[4] The method according to any one of [1] to [3], wherein the cancer is gastric cancer.
[5] A method for identifying a patient with cancer or a subject with a risk of cancer, in whom a substance suppressing the expression and/or activity of a polypeptide encoded by a fusion polynucleotide produced by a gene fusion serving as a responsible mutation (driver mutation) for cancer shows a therapeutic effect, the method comprising the steps of:
(1) detecting a fusion polynucleotide of any one of (a) to (c) below, or a polypeptide encoded thereby, in an isolated sample from the subject:

(a) an ATG3-EPHB1 fusion polynucleotide which encodes a polypeptide comprising the kinase domain of EPHB1 and having kinase activity,

(b) a TNIK-RNF123 fusion polynucleotide which encodes a polypeptide comprising the kinase domain of TNIK and the SPRY domain and RING finger domain of RNF123, and having kinase activity, and

(c) an SLC12A2-NRG2 fusion polynucleotide which encodes a polypeptide comprising the AA_permease_N super family domain of SLC12A2 and the neuregulin family conserved region of NRG2, and having intracellular signaling-enhancing activity; and

(2) determining that the substance suppressing the expression and/or activity of the polypeptide shows a therapeutic effect in the subject, in the case where the fusion polynucleotide of any one of (a) to (c) or the polypeptide encoded thereby is detected.
[6] A kit for detecting a gene fusion serving as a responsible mutation (driver mutation) for cancer, the kit comprising any or a combination of (A) to (C) below:
(A) a polynucleotide that serves as a probe designed to specifically recognize an ATG3-EPHB1 fusion polynucleotide, a TNIK-RNF123 fusion polynucleotide, or an SLC12A2-NRG2 fusion polynucleotide;
(B) polynucleotides that serve as a pair of primers designed to enable specific amplification of an ATG3-EPHB1 fusion polynucleotide, a TNIK-RNF123 fusion polynucleotide, or an SLC12A2-NRG2 fusion polynucleotide; and
(C) an antibody that specifically recognizes an ATG3-EPHB1 fusion polypeptide, a TNIK-RNF123 fusion polypeptide, or an SLC12A2-NRG2 fusion polypeptide.
[7] An isolated ATG3-EPHB1 fusion polypeptide or a fragment thereof, which comprises the kinase domain of EPHB1 and has kinase activity.
[8] An isolated TNIK-RNF123 fusion polypeptide or a fragment thereof, which comprises the kinase domain of TNIK and the SPRY domain and RING finger domain of RNF123, and has kinase activity.
[9] An isolated SLC12A2-NRG2 fusion polypeptide or a fragment thereof, which comprises the AA_permease_N super family domain of SLC12A2 and the neuregulin family conserved region of NRG2, and has intracellular signaling-enhancing activity.
[10] A polynucleotide encoding the fusion polypeptide or the fragment thereof according to any one of [7] to [9].
[11] A therapeutic agent for cancer positive for ATG3-EPHB1, TNIK-RNF123 or SLC12A2-NRG2 gene fusion.
[12] The therapeutic agent according to [11], comprising, as an active ingredient, a substance suppressing the expression and/or activity of: an ATG3-EPHB1 fusion polypeptide comprising the kinase domain of EPHB1 and having kinase activity; a TNIK-RNF123 fusion polypeptide comprising the kinase domain of TNIK and the SPRY domain and RING finger domain of RNF123 and having kinase activity; or an SLC12A2-NRG2 fusion polypeptide comprising the AA_permease_N super family domain of SLC12A2 and the neuregulin family conserved region of NRG2, and having intracellular signaling-enhancing activity.
[13] The therapeutic agent according to [11] or [12], wherein the cancer is gastric cancer.
[14] A drug for use in the treatment of cancer, the drug comprising a substance suppressing the expression and/or activity of: an ATG3-EPHB1 fusion polypeptide comprising the kinase domain of EPHB1 and having kinase activity; a TNIK-RNF123 fusion polypeptide comprising the kinase domain of TNIK and the SPRY domain and RING finger domain of RNF123 and having kinase activity; or an SLC12A2-NRG2 fusion polypeptide comprising the AA_permease_N super family domain of SLC12A2 and the neuregulin family conserved region of NRG2, and having intracellular signaling-enhancing activity.
[15] The drug according to [14], wherein the cancer is cancer positive for ATG3-EPHB1, TNIK-RNF123 or SLC12A2-NRG2 gene fusion.
[16] The drug according to [14] or [15], wherein the cancer is gastric cancer.
[17] A method for treatment of cancer, comprising the step of administering to a subject a therapeutically effective amount of a substance suppressing the expression and/or activity of: an ATG3-EPHB1 fusion polypeptide comprising the kinase domain of EPHB1 and having kinase activity; a TNIK-RNF123 fusion polypeptide comprising the kinase domain of TNIK and the SPRY domain and RING finger domain of RNF123 and having kinase activity; or an SLC12A2-NRG2 fusion polypeptide comprising the AA_permease_N super family domain of SLC12A2 and the neuregulin family conserved region of NRG2, and having intracellular signaling-enhancing activity.
[18] The method according to [17], wherein the subject is a patient with cancer positive for ATG3-EPHB1, TNIK-RNF123 or SLC12A2-NRG2 gene fusion.
[19] The method according to [17], wherein the subject is a patient with gastric cancer.
[20] A method for screening a cancer therapeutic agent, the method comprising the steps of:
(1) bringing a test substance into contact with a cell that expresses: an ATG3-EPHB1 fusion polypeptide comprising the kinase domain of EPHB1 and having kinase activity; a TNIK-RNF123 fusion polypeptide comprising the kinase domain of TNIK and the SPRY domain and RING finger domain of RNF123 and having kinase activity; or an SLC12A2-NRG2 fusion polypeptide comprising the AA_permease_N super family domain of SLC12A2 and the neuregulin family conserved region of NRG2, and having intracellular signaling-enhancing activity;
(2) determining whether the expression and/or activity of any of said fusion polypeptides is suppressed or not; and
(3) selecting the substance determined to suppress the expression and/or activity of any of said fusion polypeptides, as a cancer therapeutic agent.

### Advantageous Effects of Invention

The present invention makes it possible to detect unknown responsible mutations for the particular cancer, which have been first identified according to the present invention; to identify, based on the presence of said responsible mutations, patients with said cancer or subjects with a risk of said cancer, in whom a cancer treatment takes effect; and to treat said patients. In other words, this invention provides stratification biomarkers for use in personalized cancer medicine as well as new targets for molecular targeted therapies against cancers.

### BRIEF DESCRIPTION OF DRAWINGS

FIG. 1 is a schematic drawing showing examples of the genome structures of ATG3 and EPHB1, and the domain structure of an ATG3-EPHB1 fusion polypeptide (SEQ ID NO: 2).

FIG. 2 is a schematic drawing showing examples of the genome structures of TNIK and RNF123, and the domain structure of a TNIK-RNF123 fusion polypeptide (SEQ ID NO: 4).

FIG. 3 is a schematic drawing showing examples of the genome structures of SLC12A2 and NRG2, and the domain structure of an SLC12A2-NRG2 fusion polypeptide (SEQ ID NO: 28).

FIG. 4 is a schematic drawing showing examples of the genome structures of SLC12A2 and NRG2, and the domain structure of an SLC12A2-NRG2 fusion polypeptide (SEQ ID NO: 20).

### DESCRIPTION OF EMBODIMENTS

As disclosed below in Examples, the present inventors first found, in cancer tissues, the following three types of gene fusions serving as responsible mutations (driver mutations) for cancer: ATG3-EPHB1 gene fusion, TNIK-RNF123 gene fusion, and SLC12A2-NRG2 gene fusion. On the basis of this finding, the present invention mainly provides a method for detecting said gene fusions; a method for identifying, based on the presence of said responsible mutations, patients with cancer or subjects with a risk of cancer, in whom cancer treatments take effect; a method for treatment of cancer; a cancer therapeutic agent; and a method for screening a cancer therapeutic agent.

For the purpose of the present invention, the term “responsible mutations for cancer” is a term used interchangeably with the term “driver mutations”, and refers to mutations that are present in cancer tissues and which are capable of inducing carcinogenesis of cells. Typically speaking, if a mutation is found in a cancer tissue in which none of known oncogene mutations exists (in other words, if a mutation exists in a mutually exclusive manner with the known oncogene mutations), then the mutation can be determined to be a responsible mutation for cancer.

<Specific Responsible Mutations for Cancer>

Hereunder, the respective gene fusions will be explained. For the purpose of the present specification, the “point of fusion” in a fusion polynucleotide refers to a boundary that connects gene segments each extending toward the 5′- or 3′-end, in other words, a boundary between two nucleotide residues. The “point of fusion” in a fusion polypeptide refers to a boundary that connects polypeptides each extending toward the N- or C-terminus, in other words, a boundary between two amino acid residues, or, if a gene fusion occurs in one codon, one amino acid residue per se encoded by the codon.

(1) ATG3-EPHB1 Gene Fusion

This gene fusion is a mutation that expresses a fusion protein between ATG3 protein and EPHB1 protein (hereinafter also referred to as the “ATG3-EPHB1 fusion polypeptide”) and which is caused by an inversion (inv3) having breakpoints in regions 3q13.2 and 3q21-q23 of a human chromosome.

ATG3 protein is a protein encoded by the gene located on chromosome 3q13.2 in human, and is typified by a protein consisting of the amino acid sequence of SEQ ID NO: 30 (NCBI accession No. NP_071933.2 (29 Sep. 2013)). ATG3 protein is an E2-like conjugation enzyme essential for autophagy (Tanida I., Tanida-Miyake E., Komatsu M., Ueno T., Kominami E., “Human Apg3p/Aut1p homologue is an authentic E2 enzyme for multiple substrates, GATE-16, GABARAP, and MAP-LC3, and facilitates the conjugation of hApg12p to hApg5p.”, J Biol Chem., 19 Apr. 2002; 277 (16): 13739-44).

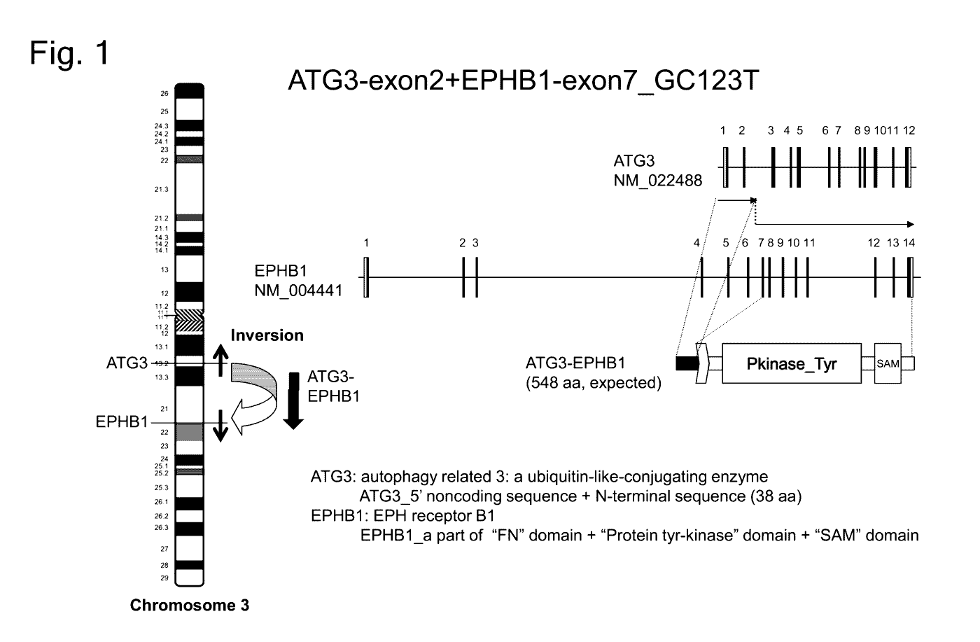

EPHB1 protein is a protein encoded by the gene located on chromosome 3q21-q23 in human, and is typified by a protein consisting of the amino acid sequence of SEQ ID NO: 32 (NCBI accession No. NP_004432.1 (25 Aug. 2013)). EPHB1 protein is a transmembrane-ephrin receptor protein belonging to the receptor tyrosine kinase family (Vindis C., Cerretti D. P., Daniel T. O., Huynh-Do U., “EphB1 recruits c-Src and p52Shc to activate MAPK/ERK and promote chemotaxis.”, J Cell Biol., 18 Aug. 2003; 162 (4): 661-71). EPHB1 protein is characterized by having the kinase domain (FIG. 1), which corresponds to, for example, the amino acid sequence at positions 619 to 878 of the amino acid sequence of SEQ ID NO: 32 in humans.

In EPHB1 protein, a SAM domain (e.g., positions 908 to 975 of the amino acid sequence of SEQ ID NO: 32) is present in a region toward the C-terminus relative to the kinase domain.

The ATG3-EPHB1 fusion polypeptide is a polypeptide comprising the kinase domain of EPHB1 protein and having kinase activity.

The ATG3-EPHB1 fusion polypeptide can contain all of the kinase domain of EPHB1 protein, or can contain part of said kinase domain as long as the ATG3-EPHB1 fusion polypeptide has kinase activity.

The expression saying that the ATG3-EPHB1 fusion polypeptide “has kinase activity” means that said fusion polypeptide is active as an enzyme phosphorylating tyrosine due to the kinase domain derived from EPHB1 protein. The kinase activity of the ATG3-EPHB1 fusion polypeptide is determined by a conventional method, and is commonly determined by incubating the fusion polypeptide with a substrate (e.g., synthetic peptide substrate) and ATP under appropriate conditions and then detecting phosphorylated tyrosine in the substrate. The kinase activity can also be determined using a commercially available measurement kit.

The ATG3 protein-derived sequence present in the ATG3-EPHB1 fusion polypeptide is not particularly limited.

In the present invention, the polynucleotide encoding the ATG3-EPHB1 fusion polypeptide (hereinafter also referred to as the “ATG3-EPHB1 fusion polynucleotide”) is a polynucleotide that encodes a polypeptide comprising the kinase domain of EPHB1 protein and having kinase activity. The ATG3-EPHB1 fusion polynucleotide can be in the form of any of mRNA, cDNA and genomic DNA.

The ATG3-EPHB1 fusion polynucleotide according to the present invention can be, for example, an ATG3-EPHB1 fusion polynucleotide encoding the polypeptide mentioned below:

(i) a polypeptide consisting of the amino acid sequence of SEQ ID NO: 2,
(ii) a polypeptide consisting of an amino acid sequence derived from the amino acid sequence of SEQ ID NO: 2 by deletion, substitution or addition of one or more amino acids, and the polypeptide having kinase activity, or
(iii) a polypeptide consisting of an amino acid sequence having a sequence identity of at least 80% to the amino acid sequence of SEQ ID NO: 2, and the polypeptide having kinase activity.

As disclosed below in Examples, the amino acid sequence of SEQ ID NO: 2 is an amino acid sequence encoded by the ATG3-EPHB1 fusion polynucleotide which was found in a sample from a human cancer tissue.

As used above in (ii), the phrase “one or more amino acids” refers to generally 1 to 50 amino acids, preferably 1 to 30 amino acids, more preferably 1 to 10 amino acids, still more preferably one to several amino acids (for example, 1 to 5 amino acids, 1 to 4 amino acids, 1 to 3 amino acids, 1 or 2 amino acids, or one amino acid).

As used above in (iii), the “sequence identity of at least 80%” refers to a sequence identity of preferably at least 85%, more preferably at least 90% or at least 95%, still more preferably at least 97%, at least 98% or at least 99%. Amino acid sequence identity can be determined using the BLASTX or BLASTP program (Altschul S. F., et al., J. Mol. Biol., 1990, 215: 403) which is based on the BLAST algorithm developed by Karlin and Altschul (Proc. Natl. Acad. Sci. USA, 1990, 87: 2264-2268; and Proc. Natl. Acad. Sci. USA, 1993, 90: 5873). In the process of making amino acid sequence analysis using BLASTX, the parameter setting is typically made as follows: score=50 and wordlength=3. In the process of making amino acid sequence analysis using the BLAST and Gapped BLAST programs, the default parameters of these programs are used. The specific procedures for conducting these analyses are well known to those skilled in the art (e.g., http://www.ncbi.nlm.nih.gov/).

Also, the ATG3-EPHB1 fusion polynucleotide according to the present invention can be, for example, any one of the polynucleotides mentioned below:

(i) a polynucleotide consisting of the nucleotide sequence of SEQ ID NO: 1,
(ii) a polynucleotide that hybridizes under stringent conditions with a polynucleotide consisting of a nucleotide sequence complementary to the polynucleotide consisting of the nucleotide sequence of SEQ ID NO: 1, and which encodes a polypeptide having kinase activity,
(iii) a polynucleotide that consists of a nucleotide sequence derived from the nucleotide sequence of SEQ ID NO: 1 by deletion, substitution or addition of one or more nucleotides, and which encodes a polypeptide having kinase activity, or
(iv) a polynucleotide that has a sequence identity of at least 80% to the polynucleotide consisting of the nucleotide sequence of SEQ ID NO: 1, and which encodes a polypeptide having kinase activity.

As disclosed below in Examples, the nucleotide sequence of SEQ ID NO: 1 is a nucleotide sequence of an ATG3-EPHB1 fusion polynucleotide which was found in a sample from a human cancer tissue.

As used above in (ii), the phrase “under stringent conditions” refers to moderately or highly stringent conditions, unless particularly specified.

The moderately stringent conditions can be easily designed by those skilled in the art on the basis of, for example, the length of a polynucleotide of interest. Basic conditions are described in Sambrook, et al., Molecular Cloning: A Laboratory Manual, 3rd ed., ch. 6-7, Cold Spring Harbor Laboratory Press, 2001. Typically, the moderately stringent conditions comprise: prewashing of a nitrocellulose filter in 5×SSC, 0.5% SDS, 1.0 mM EDTA (pH 8.0); hybridization in ca. 50% formamide, 2-6×SSC at about 40-50° C. (or any other similar hybridization solution like a Stark's solution in ca. 50% formamide at about 42° C.); and washing of the filter in 0.5-6×SSC, 0.1% SDS at about 40° C.-60° C. The moderately stringent conditions preferably comprise hybridization in 6×SSC at about 50° C., and may comprise the prewashing and/or washing under the above-mentioned conditions.

The highly stringent conditions can also be easily designed by those skilled in the art on the basis of, for example, the length of a polynucleotide of interest. The highly stringent conditions involve a higher temperature and/or a lower salt concentration than the moderately stringent conditions. Typically, the highly stringent conditions comprise hybridization in 0.2-6×SSC, preferably 6×SSC, more preferably 2×SSC, still more preferably 0.2×SSC, at about 65° C. In any case, the highly stringent conditions preferably comprise washing in 0.2×SSC, 0.1% SDS at about 65-68° C.

In any case, as a buffer for use in hybridization, prewashing and washing, SSPE (1×SSPE: 0.15 M NaCl, 10 mM NaH₂PO₄, and 1.25 mM EDTA, pH 7.4) can be used in place of SSC (1×SSC: 0.15 M NaCl and 15 mM sodium citrate). In any case, washing can be done for about 15 minutes after the completion of hybridization.

As used above in (iii), the phrase “one or more nucleotides” refers to generally 1 to 50 nucleotides, preferably 1 to 30 nucleotides, more preferably 1 to 10 nucleotides, still more preferably one to several nucleotides (for example, 1 to 5 nucleotides, 1 to 4 nucleotides, 1 to 3 nucleotides, 1 or 2 nucleotides, or one nucleotide).

As used above in (iv), the “sequence identity of at least 80%” refers to a sequence identity of preferably at least 85%, more preferably at least 90% or at least 95%, still more preferably at least 97%, at least 98% or at least 99%. Nucleotide sequence identity can be determined using the BLASTN program (Altschul S. F., et al., J. Mol. Biol., 1990, 215: 403) which is based on the above-mentioned BLAST algorithm. In the process of making nucleotide sequence analysis using BLASTN, the parameter setting is typically made as follows: score=100 and wordlength=12.

(2) TNIK-RNF123 Gene Fusion

This gene fusion is a mutation that expresses a fusion protein between TNIK protein and RNF123 protein (hereinafter also referred to as the “TNIK-RNF123 fusion polypeptide”) and which is caused by an inversion (inv3) having breakpoints in regions 3q26.31 and 3p24.3 of a human chromosome.

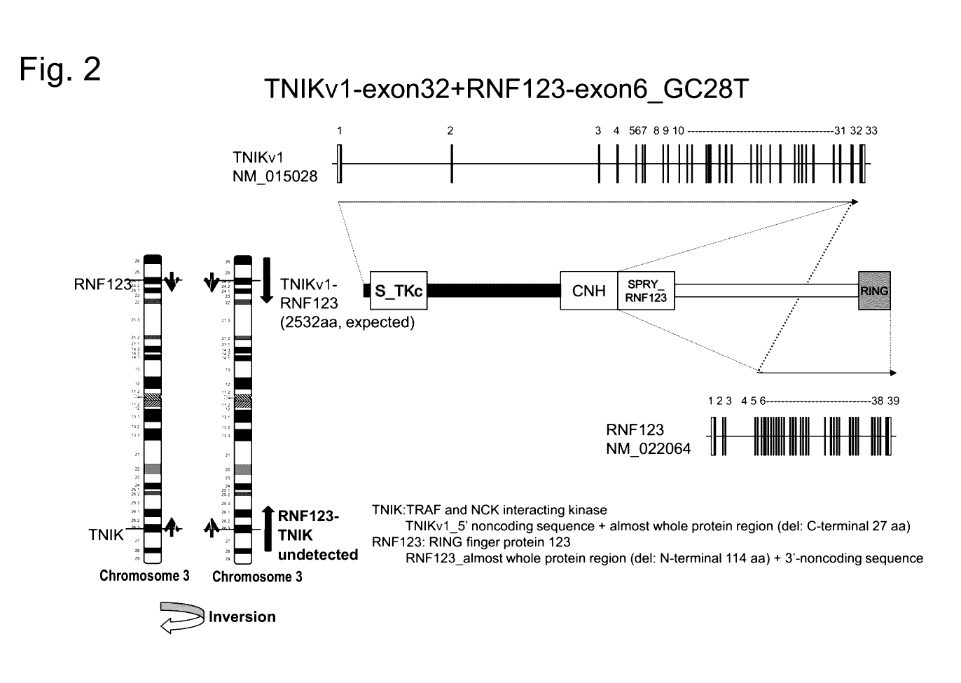

TNIK protein is a protein encoded by the gene located on chromosome 3q26.31 in human, and is typified by a protein consisting of the amino acid sequence of SEQ ID NO: 34, 36, 38, 40, 42, 44, 46 or 48 (NCBI accession No. NP_055843.1 (17 Apr. 2013), NP_001155032.1 (17 Apr. 2013), NP_001155033.1 (17 Apr. 2013), NP_001155034.1 (17 Apr. 2013), NP_001155035.1 (17 Apr. 2013), NP_001155036.1 (17 Apr. 2013), NP_001155037.1 (17 Apr. 2013), or NP_001155038.1 (17 Apr. 2013)). TNIK protein has the serine/threonine kinase domain at the N-terminus and the CNH (GCK) domain at the C-terminus (FIG. 2), and is known to be involved, for example, in cytoskeleton regulation (Fu C. A., Shen M., Huang B. C., Lasaga J., Payan D. G., Luo Y., “TNIK, a novel member of the germinal center kinase family that activates the c-Jun N-terminal kinase pathway and regulates the cytoskeleton.”, J Biol Chem., 22 Oct. 1999; 274 (43): 30729-37) and in Wnt signaling activation (Mahmoudi T., Li V. S., Ng S. S., Taouatas N., Vries R. G., Mohammed S., Heck A. J., Clevers H., “The kinase TNIK is an essential activator of Wnt target genes.”, EMBO J., 4 Nov. 2009; 28 (21): 3329-40). The kinase domain corresponds to, for example, the amino acid sequence at positions 25 to 289 of the amino acid sequence of SEQ ID NO: 34 in humans. The CNH domain corresponds to, for example, the amino acid sequence at positions 1042 to 1340 of the amino acid sequence of SEQ ID NO: 34 in humans.

RNF123 protein is a protein encoded by the gene located on chromosome 3p24.3 in human, and is typified by a protein consisting of the amino acid sequence of SEQ ID NO: 50 (NCBI accession No. NP_071347.2 (26 Aug. 2013)). RNF123 protein is an E3 ubiquitin ligase enzyme which ubiquitinates cyclin-dependent kinase inhibitor 1B (CDKN1B=p27, Kip1) (Kamura T., Hara T., Matsumoto M., Ishida N., Okumura F., Hatakeyama S., Yoshida M., Nakayama K., Nakayama K. I., “Cytoplasmic ubiquitin ligase KPC regulates proteolysis of p27(Kip1) at G1 phase.”, Nat Cell Biol., December 2004; 6 (12): 1229-35). RNF123 protein is characterized by having the SPRY domain and the RING finger domain (FIG. 2). The SPRY domain corresponds to, for example, the amino acid sequence at positions 132 to 251 of the amino acid sequence of SEQ ID NO: 50 in humans. The RING finger domain corresponds to, for example, the amino acid sequence at positions 1254 to 1295 of the amino acid sequence of SEQ ID NO: 50 in humans.

The TNIK-RNF123 fusion polypeptide is a polypeptide comprising the kinase domain of TNIK protein and the SPRY domain and RING finger domain of RNF123 and having kinase activity.

The TNIK-RNF123 fusion polypeptide can contain all of the kinase domain of TNIK protein, or can contain part of said kinase domain as long as the TNIK-RNF123 fusion polypeptide has kinase activity.

The expression saying that the TNIK-RNF123 fusion polypeptide “has kinase activity” means that said fusion polypeptide is active as an enzyme phosphorylating serine or threonine due to the kinase domain derived from TNIK protein. The kinase activity of the TNIK-RNF123 fusion polypeptide is determined by a conventional method, and is commonly determined by incubating the fusion polypeptide with a substrate (e.g., synthetic peptide substrate) and ATP under appropriate conditions and then detecting phosphorylated serine or threonine in the substrate. The kinase activity can also be determined using a commercially available measurement kit.

The TNIK-RNF123 fusion polypeptide can contain all of the SPRY domain and the RING finger domain of RNF123 protein, or can contain part of the SPRY domain and the RING finger domain as long as their original functions in RNF123 protein can be retained. The expression saying that part of the SPRY domain and the RING finger domain retains their original functions in RNF123 protein means that RNF123 protein containing part of the SPRY domain and RING finger domain has E3 activity. The E3 activity can be determined by, for example, a method described in Kamura, T., et al., Nature Cell Biology, 2004, 6 (12), 1229-35.

In the present invention, the polynucleotide encoding the TNIK-RNF123 fusion polypeptide (hereinafter also referred to as the “TNIK-RNF123 fusion polynucleotide”) is a polynucleotide that encodes the polypeptide comprising the kinase domain of TNIK protein and the SPRY domain and RING finger domain of RNF123, and having kinase activity. The TNIK-RNF123 fusion polynucleotide can be in the form of any of mRNA, cDNA and genomic DNA.

The TNIK-RNF123 fusion polynucleotide according to the present invention can be, for example, a TNIK-RNF123 fusion polynucleotide encoding the polypeptide mentioned below:

(i) a polypeptide consisting of the amino acid sequence of SEQ ID NO: 4, 6, 8, 10, 12, 14, 16 or 18,
(ii) a polypeptide consisting of an amino acid sequence derived from the amino acid sequence of SEQ ID NO: 4, 6, 8, 10, 12, 14, 16 or 18 by deletion, substitution or addition of one or more amino acids, and the polypeptide having kinase activity, or
(iii) a polypeptide consisting of an amino acid sequence having a sequence identity of at least 80% to the amino acid sequence of SEQ ID NO: 4, 6, 8, 10, 12, 14, 16 or 18, and the polypeptide having kinase activity.

As disclosed below in Examples, the amino acid sequence of SEQ ID NO: 4, 6, 8, 10, 12, 14, 16 or 18 is an amino acid sequence encoded by the TNIK-RNF123 fusion polynucleotide comprising partial sequences, which was found in a sample from a human cancer tissue.

The phrases “one or more amino acids” and “sequence identity of at least 80%” as used above in (ii) and (iii), respectively, have the same meanings as described above in “(1) ATG3-EPHB1 gene fusion”.

Also, the TNIK-RNF123 fusion polynucleotide according to the present invention can be, for example, any one of the polynucleotides mentioned below:

(i) a polynucleotide consisting of the nucleotide sequence of SEQ ID NO: 3, 5, 7, 9, 11, 13, 15 or 17,
(ii) a polynucleotide that hybridizes under stringent conditions with a polynucleotide consisting of a nucleotide sequence complementary to the polynucleotide consisting of the nucleotide sequence of SEQ ID NO: 3, 5, 7, 9, 11, 13, 15 or 17, and which encodes a polypeptide having kinase activity,
(iii) a polynucleotide that consists of a nucleotide sequence derived from the nucleotide sequence of SEQ ID NO: 3, 5, 7, 9, 11, 13, 15 or 17 by deletion, substitution or addition of one or more nucleotides, and which encodes a polypeptide having kinase activity, or
(iv) a polynucleotide that has a sequence identity of at least 80% to the polynucleotide consisting of the nucleotide sequence of SEQ ID NO: 3, 5, 7, 9, 11, 13, 15 or 17, and which encodes a polypeptide having kinase activity.

As disclosed below in Examples, the nucleotide sequence of SEQ ID NO: 3 is a nucleotide sequence of a TNIK-RNF123 fusion polynucleotide comprising partial sequences, which was found in a sample from a human cancer tissue.

The phrases “under stringent conditions”, “one or more nucleotides”, and “sequence identity of at least 80%” as used above in (ii), (iii) and (iv), respectively, have the same meanings as described above in “(1) ATG3-EPHB1 gene fusion”.

(3) SLC12A2-NRG2 Gene Fusion

This gene fusion is a mutation that expresses a fusion protein between SLC12A2 protein and NRG2 protein (hereinafter also referred to as the “SLC12A2-NRG2 fusion polypeptide”) and which is caused by an inversion (inv5) having breakpoints in regions 5q23.3 and 5q23-q33 of a human chromosome.

SLC12A2 protein is a protein encoded by the gene located on chromosome 5q23.3 in humans, and is typified by a protein consisting of the amino acid sequence of SEQ ID NO: 52 or 54 (NCBI accession No. NP_001037.1 (22 Sep. 2013) or NP_001243390.1 (28 Sep. 2013)). SLC12A2 protein is a membrane protein functioning as a Na—K—Cl cotransporter (Somasekharan S., Tanis J., Forbush B., “Loop diuretic and ion-binding residues revealed by scanning mutagenesis of transmembrane helix 3 (TM3) of Na—K—Cl cotransporter (NKCC1).”, J Biol Chem., 18 May 2012; 287 (21): 17308-17), and having the AA_permease_N super family domain at the N-terminus (FIG. 1). This domain corresponds to, for example, the amino acid sequence at positions 210 to 267 of the amino acid sequence of SEQ ID NO: 52 in humans.

NRG2 protein is a protein encoded by the gene located on chromosome 5q23-q33 in humans, and is typified by a protein consisting of the amino acid sequence of SEQ ID NO: 56, 58, 60, 62 or 64 (NCBI accession No. NP_004874.1 (25 Aug. 2013), NP_053584.1 (25 Aug. 2013), NP_053585.1 (25 Aug. 2013), NP_053586.1 (25 Aug. 2013) or NP_001171864.1 (25 Aug. 2013)). NRG2 protein is a protein belonging to the neuregulin family which controls cell growth and differentiation through interaction with ERBB family receptors (Chang H., Riese D. J. 2nd, Gilbert W., Stern D. F., McMahan U. J., “Ligands for ErbB-family receptors encoded by a neuregulin-like gene.”, Nature, 29 May 1997; 387 (6632): 509-12; Carraway K. L. 3rd, Weber J. L., Unger M. J., Ledesma J., Yu N., Gassmann M., Lai C., “Neuregulin-2, a new ligand of ErbB3/ErbB4-receptor tyrosine kinases.”, Nature, 29 May 1997; 387 (6632): 512-6; Hobbs S. S., Coffing S. L., Le A. T., Cameron E. M., Williams E. E., Andrew M., Blommel E. N., Hammer R. P., Chang H., Riese D. J. 2nd., “Neuregulin isoforms exhibit distinct patterns of ErbB family receptor activation.”, Oncogene, 5 Dec. 2002; 21 (55): 8442-52; and Nakano N., Higashiyama S., Ohmoto H., Ishiguro H., Taniguchi N., Wada Y., “The N-terminal region of NTAK/neuregulin-2 isoforms has an inhibitory activity on angiogenesis.”, J Biol Chem., 19 Mar. 2004; 279 (12): 11465-70.). NRG2 protein is characterized by having the neuregulin family conserved region (FIG. 1). This domain corresponds to, for example, the amino acid sequence at positions 398 to 718 of the amino acid sequence of SEQ ID NO: 56 in humans.

In NRG2 protein, the I-set domain (e.g., positions 239 to 328 of the amino acid sequence of SEQ ID NO: 56) is present in a region toward the N-terminus relative to the neuregulin family conserved region. Further, there is another isoform having the EGF domain (e.g., positions 353 to 381 of the amino acid sequence of SEQ ID NO: 56) between the I-set domain and the neuregulin family conserved region.

The SLC12A2-NRG2 fusion polypeptide is a polypeptide comprising the AA_permease_N super family domain of SLC12A2 protein and the neuregulin family conserved region of NRG2 protein, and having intracellular signaling-enhancing activity.

The SLC12A2-NRG2 fusion polypeptide can contain all of the AA_permease_N super family domain of SLC12A2 protein, or can contain part of the AA_permease_N super family domain as long as the SLC12A2-NRG2 fusion polypeptide has intracellular signaling-enhancing activity.

The SLC12A2-NRG2 fusion polypeptide can contain all of the neuregulin family conserved region of NRG2 protein, or can contain part of the neuregulin family conserved region as long as the SLC12A2-NRG2 fusion polypeptide has intracellular signaling-enhancing activity.

The expression saying that the SLC12A2-NRG2 fusion polypeptide “has intracellular signaling-enhancing activity” means that said fusion polypeptide is active in enhancing intracellular signaling due to the neuregulin family conserved region derived from NRG2 protein. This activity is determined by the following method described in Wilson, T. R., et al., Cancer Cell, 2011, 20, 158-172.

A test substance is added to EFM-19 cells (DSMZ, No. ACC-231) cultured in a serum-starved condition, the cells are treated for 30 minutes and then lysed to extract protein. The phosphorylation of EGFR, ERBB2, ERBB3 or ERBB4 is analyzed by Western blotting. If phosphorylation is higher than in the case where no test substance is added, it is determined that the test substance has intracellular signaling-enhancing activity. As the test substance as referred to herein, the entire SLC12A2-NRG2 fusion polypeptide may be used, or a fragment thereof which lacks a transmembrane domain but contains the neuregulin family conserved region may be used in consideration of solubility or other factors.

The SLC12A2-NRG2 fusion polypeptide can contain all or part of the I-set domain of NRG2 protein as long as said fusion polypeptide has intracellular signaling-enhancing activity.

The SLC12A2-NRG2 fusion polypeptide can contain all or part of the EGF domain of NRG2 protein as long as said fusion polypeptide has intracellular signaling-enhancing activity.

In the present invention, the polynucleotide encoding the SLC12A2-NRG2 fusion polypeptide (hereinafter also referred to as the “SLC12A2-NRG2 fusion polynucleotide”) is a polynucleotide that encodes the polypeptide comprising the AA_permease_N super family domain of SLC12A2 protein and the neuregulin family conserved region of NRG2 protein, and having intracellular signaling-enhancing activity. The SLC12A2-NRG2 fusion polynucleotide can be in the form of any of mRNA, cDNA and genomic DNA.

The SLC12A2-NRG2 fusion polynucleotide according to the present invention can be, for example, an SLC12A2-NRG2 fusion polynucleotide encoding the polypeptide mentioned below:

(i) a polypeptide consisting of the amino acid sequence of SEQ ID NO: 20, 22, 24, 26 or 28,
(ii) a polypeptide consisting of an amino acid sequence derived from the amino acid sequence of SEQ ID NO: 20, 22, 24, 26 or 28 by deletion, substitution or addition of one or more amino acids, and the polypeptide having intracellular signaling-enhancing activity, or
(iii) a polypeptide consisting of an amino acid sequence having a sequence identity of at least 80% to the amino acid sequence of SEQ ID NO: 20, 22, 24, 26 or 28, and the polypeptide having intracellular signaling-enhancing activity.

As disclosed below in Examples, the amino acid sequence of SEQ ID NO: 20, 22, 24, 26 or 28 is an amino acid sequence encoded by the SLC12A2-NRG2 fusion polynucleotide comprising partial sequences, which was found in a sample from a human cancer tissue.

The phrases “one or more amino acids” and “sequence identity of at least 80%” as used above in (ii) and (iii), respectively, have the same meanings as described above in “(1) ATG3-EPHB1 gene fusion”.

Also, the SLC12A2-NRG2 fusion polynucleotide according to the present invention can be, for example, any one of the polynucleotides mentioned below:

(i) a polynucleotide consisting of the nucleotide sequence of SEQ ID NO: 19, 21, 23, 25 or 27,
(ii) a polynucleotide that hybridizes under stringent conditions with a polynucleotide consisting of a nucleotide sequence complementary to the polynucleotide consisting of the nucleotide sequence of SEQ ID NO: 19, 21, 23, 25 or 27, and which encodes a polypeptide having intracellular signaling-enhancing activity,
(iii) a polynucleotide that consists of a nucleotide sequence derived from the nucleotide sequence of SEQ ID NO: 19, 21, 23, 25 or 27 by deletion, substitution or addition of one or more nucleotides, and which encodes a polypeptide having intracellular signaling-enhancing activity, or
(iv) a polynucleotide that has a sequence identity of at least 80% to the polynucleotide consisting of the nucleotide sequence of SEQ ID NO: 19, 21, 23, 25 or 27, and which encodes a polypeptide having intracellular signaling-enhancing activity.

As disclosed below in Examples, the nucleotide sequence of SEQ ID NO: 19, 21, 23, 25 or 27 is a nucleotide sequence of an SLC12A2-NRG2 fusion polynucleotide comprising partial sequences, which was found in a sample from a human cancer tissue.

The phrases “under stringent conditions”, “one or more nucleotides”, and “sequence identity of at least 80%” as used above in (ii), (iii) and (iv), respectively, have the same meanings as described above in “(1) ATG3-EPHB1 gene fusion”.

<Method for Detecting Gene Fusions Serving as Responsible Mutations (Driver Mutations) for Cancer>

The present invention provides a method for detecting the above-mentioned three types of gene fusions (hereinafter also referred to as the “inventive detection method”). The inventive detection method comprises the step of detecting any one of the above-mentioned fusion polynucleotides, or a polypeptide encoded thereby, in an isolated sample from a subject.

In the inventive detection method, the subject is not particularly limited as long as it is a mammal. Examples of the mammal include: rodents such as mouse, rat, hamster, chipmunk and guinea pig; rabbit, pig, cow, goat, horse, sheep, mink, dog, cat; and primates such as human, monkey, cynomolgus monkey, rhesus monkey, marmoset, orangutan, and chimpanzee, with human being preferred.

The subject may be not only a subject affected by cancer, but also a subject suspected of being affected by cancer or a subject with a future risk of cancer. The “cancer” to which the inventive detection method is to be applied is not particularly limited as long as it is a cancer in which any of the above-mentioned three types of gene fusions can be detected, with gastric cancer being preferred, and special-type gastric cancer, undifferentiated gastric cancer, or moderately differentiated gastric cancer being more preferred.

The “isolated sample” from the subject encompasses not only biological samples (for example, cells, tissues, organs (e.g., small intestine, spleen, kidney, liver, stomach, lung, adrenal gland, heart, brain, pancreas, and aorta), body fluids (e.g., blood, lymphs, bone marrow fluid), digestive juices, sputum, bronchioalveolar/bronchial lavage fluids, urine, and feces), but also nucleic acid extracts from these biological samples (e.g., genomic DNA extracts, mRNA extracts, and cDNA and cRNA preparations from mRNA extracts) and protein extracts. The genomic DNA, mRNA, cDNA or protein can be prepared by those skilled in the art through considering various factors including the type and state of the sample and selecting a known technique suitable therefor. The sample may also be the one that is fixed with formalin or alcohol, frozen, or embedded in paraffin.

Further, the “isolated sample” is preferably derived from an organ having or suspected of having the above-mentioned cancer, and more preferably derived from the stomach having or suspected of having the above-mentioned cancer. Examples of the “isolated sample” include, but are not limited to, a gastric tissue from a patient with gastric cancer (preferably, special-type gastric cancer, undifferentiated gastric cancer, or moderately differentiated gastric cancer) or a subject suspected of being affected by gastric cancer, as well as total RNA extracted from said gastric tissue.

In the inventive detection method, the detection of a fusion polynucleotide or a polypeptide encoded thereby can be made using a per se known technique.

If the object to be detected is a transcript from a genomic DNA (mRNA, or cDNA prepared from mRNA), a fusion polynucleotide in the form of mRNA or cDNA can be detected using, for example, RT-PCR, sequencing, TaqMan probe method, Northern blotting, dot blotting, or cDNA microarray analysis.

If the object to be detected is a genomic DNA, a fusion polynucleotide in the form of genomic DNA can be detected using, for example, in situ hybridization (ISH), genomic PCR, sequencing, TaqMan probe method, Southern blotting, or genome microarray analysis.

Any of the above-mentioned detection techniques can be used alone or in combination. For example, since the above-mentioned three types of gene fusions are believed to contribute to oncogenesis by expressing fusion polypeptides, it is also preferred that if a fusion polynucleotide in the form of genomic DNA is detected (e.g., by in situ hybridization or the like), production of a transcript or a protein should be further confirmed (e.g., by RT-PCR, immunostaining or the like).

If a fusion polynucleotide is detected by a hybridization technique (e.g., TaqMan probe method, Northern blotting, Southern blotting, dot blotting, microarray analysis, in situ hybridization (ISH)), detection can be made using a polynucleotide that serves as a probe designed to specifically recognize the fusion polynucleotide. As used herein, the phrase “specifically recognize the fusion polynucleotide” means that under stringent conditions, the probe distinguishes and recognizes the fusion polynucleotide from other polynucleotides, including wild-type genes from which to derive both segments of the fusion polynucleotide each extending from the point of fusion toward the 5′- or 3′-end.

Since biological samples (e.g., biopsy samples) obtained in the course of treatment or diagnosis are often fixed in formalin, it is preferred to use in situ hybridization in the inventive detection method, from the viewpoints that the genomic DNA to be detected is stable even when fixed in formalin and that this technique is high in detection sensitivity.

According to the in situ hybridization method, the genomic DNA (fusion polynucleotide) encoding a fusion polypeptide can be detected by hybridizing, to such a biological sample, the following polynucleotide (a) or (b) which has a chain length of at least 15 nucleotides and serves as a probe(s) designed to specifically recognize said fusion polynucleotide:

(a) a polynucleotide serving as at least one probe for each of the above-mentioned fusion genes, as selected from the group consisting of a probe that hybridizes to the nucleotide sequence of the fusion partner gene toward the 5′-end (ATG3, TNIK or SLC12A2 gene) and a probe that hybridizes to the nucleotide sequence of the fusion partner gene toward the 3′-end (EPHB1, RNF123 or NRG2 gene); or
(b) a polynucleotide serving as a probe for each of the above-mentioned fusion genes, which hybridizes to a nucleotide sequence containing the point of fusion between the fusion partner gene toward the 5′-end and the fusion partner gene toward the 3′-end.

The ATG3 gene according to the present invention, as far as it is derived from humans, is typified by a gene consisting of the DNA sequence (encoded into complementary strand) from position 112251354 to position 112280810 in the genome sequence identified in Genbank accession No. NC_000003.11.

The TNIK gene according to the present invention, as far as it is derived from humans, is typified by a gene consisting of the DNA sequence (encoded into complementary strand) from position 170780292 to position 171178197 in the genome sequence identified in Genbank accession No. NC_000003.11.

The SLC12A2 gene according to the present invention, as far as it is derived from humans, is typified by a gene consisting of the DNA sequence from position 127419483 to position 127525380 in the genome sequence identified in Genbank accession No. NC_000005.9.

The EPHB1 gene according to the present invention, as far as it is derived from humans, is typified by a gene consisting of the DNA sequence from position 134514099 to position 134979309 in the genome sequence identified in Genbank accession No. NC_000003.11.

The RNF123 gene according to the present invention, as far as it is derived from humans, is typified by a gene consisting of the DNA sequence from position 49726950 to position 49758962 in the genome sequence identified in Genbank accession No. NC_000003.11.

The NRG2 gene according to the present invention, as far as it is derived from humans, is typified by a gene consisting of the DNA sequence (encoded into complementary strand) from position 139226364 to position 139422884 in the genome sequence identified in Genbank accession No. NC_000005.9.

However, the DNA sequences of genes can change in nature (i.e., in a non-artificial way) due to their mutations and the like. Thus, such native mutants can also be encompassed by the present invention (the same applies hereinafter).

The polynucleotide mentioned in (a) according to the present invention can be of any type as far as it is capable of detecting the presence of the genomic DNA encoding a fusion polypeptide in the above-mentioned biological sample by hybridizing to a nucleotide sequence(s) targeted by said polynucleotide, i.e., the nucleotide sequence of the fusion partner gene toward the 5′-end (ATG3, TNIK or SLC12A2 gene) and/or the nucleotide sequence of the fusion partner gene toward the 3′-end (EPHB1, RNF123 or NRG2 gene); preferably, the polynucleotide (a) is any of the polynucleotides mentioned below in (a1) to (a3):

(a1) a combination of a polynucleotide that hybridizes to the nucleotide sequence of that region of the fusion partner gene toward the 5′-end which extends upstream from its breakpoint toward the 5′-end (this polynucleotide is hereinafter also referred to as the “5′ fusion partner gene probe 1”), and a polynucleotide that hybridizes to the nucleotide sequence of that region of the fusion partner gene toward the 3′-end which extends downstream from its breakpoint toward the 3′-end (this polynucleotide is hereinafter also referred to as the “3′ fusion partner gene probe 1”);
(a2) a combination of a polynucleotide that hybridizes to the nucleotide sequence of that region of the fusion partner gene toward the 5′-end which extends upstream from its breakpoint toward the 5′-end (this polynucleotide is hereinafter also referred to as the “5′ fusion partner gene probe 1”), and a polynucleotide that hybridizes to the nucleotide sequence of that region of the fusion partner gene toward the 5′-end which extends downstream from its breakpoint toward the 3′-end (this polynucleotide is hereinafter also referred to as the “5′ fusion partner gene probe 2”); and
(a3) a combination of a polynucleotide that hybridizes to the nucleotide sequence of that region of the fusion partner gene toward the 3′-end which extends upstream from its breakpoint toward the 5′-end (this polynucleotide is hereinafter also referred to as the “3′ fusion partner gene probe 2”), and a polynucleotide that hybridizes to the nucleotide sequence of that region of the fusion partner gene toward the 3′-end which extends downstream from its breakpoint toward the 3′-end (this polynucleotide is hereinafter also referred to as the “3′ fusion partner gene probe 1”).

In the context of (a1) to (a3) above, when the fusion partner gene toward the 5′-end is the ATG3 gene, the nucleotide sequence of that region of the fusion partner gene toward the 5′-end which extends upstream from its breakpoint toward the 5′-end typically contains exon 2 (at positions 508 to 549 in the nucleotide sequence of SEQ ID NO: 29) of the ATG3 gene, and its upstream region.

In the context of (a1) to (a3) above, when the fusion partner gene toward the 3′-end is the EPHB1 gene, the nucleotide sequence of that region of the fusion partner gene toward the 3′-end which extends downstream from its breakpoint toward the 3′-end typically contains exon 7 (at positions 1798 to 1960 in the nucleotide sequence of SEQ ID NO: 31) of the EPHB1 gene, and its downstream region.

In the context of (a1) to (a3) above, when the fusion partner gene toward the 5′-end is the TNIK gene, the nucleotide sequence of that region of the fusion partner gene toward the 5′-end which extends upstream from its breakpoint toward the 5′-end typically contains exon 32 (at positions 4205 to 4344 in the nucleotide sequence of SEQ ID NO: 33) of the TNIK gene, and its upstream region.

In the context of (a1) to (a3) above, when the fusion partner gene toward the 3′-end is the RNF123 gene, the nucleotide sequence of that region of the fusion partner gene toward the 3′-end which extends downstream from its breakpoint toward the 3′-end typically contains exon 6 (at positions 469 to 523 in the nucleotide sequence of SEQ ID NO: 49) of the RNF123 gene, and its downstream region.

In the context of (a1) to (a3) above, when the fusion partner gene toward the 5′-end is the SLC12A2 gene, the nucleotide sequence of that region of the fusion partner gene toward the 5′-end which extends upstream from its breakpoint toward the 5′-end typically contains exon 4 (at positions 1117 to 1212 in the nucleotide sequence of SEQ ID NO: 51) of the SLC12A2 gene, and its upstream region.

In the context of (a1) to (a3) above, when the fusion partner gene toward the 3′-end is the NRG2 gene, the nucleotide sequence of that region of the fusion partner gene toward the 3′-end which extends downstream from its breakpoint toward the 3′-end typically contains exon 2 (at positions 931 to 1102 in the nucleotide sequence of SEQ ID NO: 55) of the NRG2 gene, and its downstream region.

The polynucleotides mentioned above in (a1) can be exemplified by the polynucleotide combinations mentioned below in (a1-1) to (a1-3):

(a1-1) a combination of a polynucleotide that hybridizes to exon 2 of the ATG3 gene, and a polynucleotide that hybridizes to exon 7 of the EPHB1 gene;
(a1-2) a combination of a polynucleotide that hybridizes to exon 32 of the TNIK gene, and a polynucleotide that hybridizes to exon 6 of the RNF123 gene; and
(a1-3) a combination of a polynucleotide that hybridizes to exon 4 of the SLC12A2 gene, and a polynucleotide that hybridizes to exon 2 of the NRG2 gene.

In the present invention, the region to which the polynucleotide for use for in situ hybridization as mentioned above in (a) is to hybridize (such a region is hereinafter referred to as the “target nucleotide sequence”) is preferred to be located not more than 1000000 nucleotides away from the point of fusion between the fusion partner gene toward the 5′-end (ATG3, TNIK or SLC12A2 gene) and the fusion partner gene toward the 3′-end (EPHB1, RNF123 or NRG2 gene), from the viewpoints of specificity for the target nucleotide sequence and detection sensitivity.

In the present invention, the polynucleotide for use for in situ hybridization as mentioned above in (b) can be of any type as far as it is capable of detecting the presence of the genomic DNA encoding a fusion polypeptide in the above-mentioned biological sample by hybridizing to a nucleotide sequence targeted by said polynucleotide, i.e., a nucleotide sequence containing the point of fusion between the fusion partner gene toward the 5′-end and the fusion partner gene toward the 3′-end; and typical examples of the polynucleotide (b) are those which each hybridize to a nucleotide sequence containing the point of fusion in the nucleotide sequence of SEQ ID NO: 1, 3, 5, 7, 9, 11, 13, 15, 17, 19, 21, 23, 25 or 27.

Further, in the present invention, the polynucleotide for use for in situ hybridization as mentioned above in (a) or (b) is preferred to be a group consisting of multiple types of polynucleotides which can cover the entire target nucleotide sequence, from the viewpoints of specificity for the target nucleotide sequence and detection sensitivity. In such a case, each of the polynucleotides constituting the group has a length of at least 15 nucleotides, preferably from 100 to 1000 nucleotides.

The polynucleotide for use for in situ hybridization as mentioned above in (a) or (b) is preferably labeled with a fluorescent dye or the like for the purpose of detection. Examples of such a fluorescent dye include, but are not limited to, DEAC, FITC, R6G, TexRed, and CyS. Aside from the fluorescent dye, the polynucleotide may also be labeled with a radioactive isotope (e.g. ¹²⁵I, ¹³¹I, ³H, ¹⁴C, ³³P, ³²P), an enzyme (e.g., β-galactosidase, β-glucosidase, alkaline phosphatase, peroxidase, malate dehydrogenase), or a luminescent substance (e.g., luminol, luminol derivative, luciferin, lucigenin, 3,3′-diaminobenzidine (DAB)).

When in situ hybridization is performed using a combination of 5′ fusion partner gene probe 1 and 3′ fusion partner gene probe 1, a combination of 5′ fusion partner gene probe 1 and 5′ fusion partner gene probe 2, or a combination of 3′ fusion partner gene probe 2 and 3′ fusion partner gene probe 1, the probes of each combination are preferably labeled with different dyes. If, as the result of in situ hybridization using such a combination of probes labeled with different dyes, an overlap is observed between the signal (e.g., fluorescence) emitted from the label on 5′ fusion partner gene probe 1 and the signal emitted from the label on 3′ fusion partner gene probe 1, then it can be determined that a genomic DNA encoding a fusion polypeptide of interest has been detected successfully. Also, if a split is observed between the signal emitted from the label on 5′ fusion partner gene probe 1 and the signal emitted from the label on 5′ fusion partner gene probe 2, or between the signal emitted from the label on 3′ fusion partner gene probe 2 and the signal emitted from the label on 3′ fusion partner gene probe 1, then it can be determined that a genomic DNA encoding a fusion polypeptide of interest has been detected successfully.

Polynucleotide labeling can be effected by a known method. For example, polynucleotides can be labeled by nick translation or random priming, in which the polynucleotides are caused to incorporate substrate nucleotides labeled with a fluorescent dye or the like.

The conditions for hybridizing the polynucleotide mentioned above in (a) or (b) to the above-mentioned biological sample by in situ hybridization can vary with various factors including the length of said polynucleotide; and exemplary highly stringent hybridization conditions are 0.2×SSC at 65° C., and exemplary low stringent hybridization conditions are 2.0×SSC at 50° C. Those skilled in the art could realize comparable stringent hybridization conditions to those mentioned above, by appropriately selecting salt concentration (e.g., SSC dilution rate), temperature, and various other conditions including concentrations of surfactant (e.g., NP-40) and formamide, and pH.

Besides in situ hybridization, other examples of the method for detecting a genomic DNA encoding a fusion polypeptide of interest using the polynucleotide mentioned above in (a) or (b) include Southern blotting, Northern blotting and dot blotting. According to these methods, a fusion gene of interest is detected by hybridizing said polynucleotide (a) or (b) to a membrane in which a nucleic acid extract from the above-mentioned biological sample is transcribed. In the case of using said polynucleotide (a), if a polynucleotide that hybridizes to the nucleotide sequence of the fusion partner gene toward the 5′-end and a polynucleotide that hybridizes to the nucleotide sequence of a fusion partner gene toward the 3′-end both recognize the same band developed in a membrane, then it can be determined that a genomic DNA encoding a fusion polypeptide of interest has been detected successfully.

Additional examples of the method for detecting a genomic DNA encoding a fusion polypeptide of interest using said polynucleotide (b) include genome microarray analysis and DNA microarray analysis. According to these methods, the genomic DNA is detected by preparing an array in which said polynucleotide (b) is immobilized on a substrate and bringing the above-mentioned biological sample into contact with the polynucleotide immobilized on the array. The substrate is not particularly limited as long as it allows conversion of an oligo- or polynucleotide into a solid phase, and examples include glass plate, nylon membrane, microbeads, silicon chip, and capillary.

In the inventive detection method, it is also preferred to detect a fusion polynucleotide of interest using PCR.

PCR can be performed using polynucleotides serving as a pair of primers designed to specifically amplify a fusion polynucleotide using DNA (e.g., genomic DNA, cDNA) or RNA prepared from the above-mentioned biological sample as a template. As used herein, the phrase “specifically amplify a fusion polynucleotide” means that primers do not amplify wild-type genes from which to derive both segments of a fusion polynucleotide of interest each extending from the point of fusion toward the 5′- or 3′-end, but can amplify said fusion polynucleotide alone. It is acceptable to amplify all of the fusion polynucleotide or to amplify that part of the fusion polynucleotide which contains the point of fusion.

The “polynucleotides serving as a pair of primers” to be used for PCR or the like consist of a sense primer (forward primer) and an anti-sense primer (reverse primer) that specifically amplify a target fusion polynucleotide. The sense primer is designed from the nucleotide sequence of that region of said fusion polynucleotide which extends from the point of fusion toward the 5′-end. The anti-sense primer is designed from the nucleotide sequence of that region of said fusion polynucleotide which extends from the point of fusion toward the 3′-end. From the viewpoints of the accuracy and sensitivity of PCR detection, these primers are commonly designed such that amplified PCR products are not more than 5 kb in size. The primers can be designed as appropriate by a known method, for example, using the Primer Express® software (Applied Biosystems). The length of these polynucleotides is generally not less than 15 nucleotides (preferably not less than 16, 17, 18, 19 or 20 nucleotides, more preferably not less than 21 nucleotides) and not more than 100 nucleotides (preferably not more than 90, 80, 70, 60, 50 or 40 nucleotides, more preferably not more than 30 nucleotides).

Preferred examples of the “polynucleotides serving as a pair of primers” include: a primer set for the ATG3-EPHB1 fusion polynucleotide, which consists of a polynucleotide consisting of the nucleotide sequence of SEQ ID NO: 65 and a polynucleotide consisting of the nucleotide sequence of SEQ ID NO: 66; a primer set for the TNIK-RNF123 fusion polynucleotide, which consists of a polynucleotide consisting of the nucleotide sequence of SEQ ID NO: 67 and a polynucleotide consisting of the nucleotide sequence of SEQ ID NO: 68; and a primer set for the SLC12A2-NRG2 fusion polynucleotide, which consists of a polynucleotide consisting of the nucleotide sequence of SEQ ID NO: 69 and a polynucleotide consisting of the nucleotide sequence of SEQ ID NO: 70 (refer to Table 1 shown below).

In the process of detecting a fusion polynucleotide by PCR, direct sequencing is performed on PCR products to sequence a nucleotide sequence containing the point of fusion, whereby it can be confirmed that a gene segment toward the 5′-end and a gene segment toward the 3′-end are joined in-frame and/or that specified domains are contained in the fusion polynucleotide. Sequencing can be done by a known method—it can be easily done by using a sequencer (e.g., ABI-PRISM 310 Genetic Analyzer (Applied Biosystems Inc.)) in accordance with its operating instructions.

Also, in the process of detecting a fusion polynucleotide by PCR, it can be confirmed by the TaqMan probe method that a gene segment toward the 5′-end and a gene segment toward the 3′-end are joined in-frame and/or that specified domains are contained in the fusion polynucleotide. The probe to be used in the TaqMan probe method can be exemplified by the polynucleotide mentioned above in (a) or (b). The probe is labeled with a reporter dye (e.g., FAM, FITC, VIC) and a quencher (e.g., TAMRA, Eclipse, DABCYL, MGB).

The above-mentioned primers and probes can be in the form of DNA, RNA, or DNA/RNA chimera, and is preferably in the form of DNA. Alternatively, the primers and probes may be the ones whose nucleotides are partially or wholly substituted with an artificial nucleic acid such as PNA (polyamide nucleic acid: a peptide nucleic acid), LNA™ (Locked Nucleic Acid; a bridged nucleic acid), ENA® (2′-0,4′-C-Ethylene-bridged Nucleic Acid), GNA (glycerol nucleic acid) or TNA (threose nucleic acid). Further, the primers and probes can be double- or single-stranded, and are preferably single-stranded.

As far as the primers and probes are capable of specifically hybridizing to a target sequence, they may contain one or more nucleotide mismatches, generally have at least 80% identity, preferably at least 90%, at least 91%, at least 92%, at least 93%, or at least 94% identity, more preferably at least 95%, at least 96%, at least 97%, at least 98%, or at least 99% identity, and most preferably 100% identity, to a sequence complementary to the target sequence.

The primers and probes can be synthesized, for example, according to a conventional method using an automatic DNA/RNA synthesizer on the basis of the information on the nucleotide sequences disclosed in the present specification.

In the inventive detection method, it is also acceptable to detect a fusion polynucleotide of interest by whole-transcriptome sequencing (RNA sequencing) or genome sequencing. These techniques can be carried out, for example, using a next-generation sequencer (e.g., Genome Analyzer IIx (Illumina), HiSeq sequencer (HiSeq 2000, Illumina), Genome Sequencer FLX System (Roche)), or the like according to the manufacturer's instructions. RNA sequencing can be done by, for example, preparing a cDNA library from a total RNA using a commercially available kit (e.g., mRNA-Seq sample preparation kit (Illumina)) according to the manufacturer's instructions and sequencing the prepared library using a next-generation sequencer.

In the inventive detection method, if the object to be detected is a translation product of a fusion polynucleotide (i.e., fusion polypeptide), the translation product can be detected using, for example, immunostaining, Western blotting, RIA, ELISA, flow cytometry, immunoprecipitation, or antibody array analysis. These techniques use an antibody that specifically recognizes a fusion polypeptide. As used herein, the phrase “specifically recognizes a fusion polypeptide” means that besides the fusion polypeptide, an antibody recognizes no other proteins, including wild-type proteins from which to derive both segments of said fusion polynucleotide each extending from the point of fusion toward the N- or C-terminus. The antibody that “specifically recognizes a fusion polypeptide”, which is to be used in the inventive detection method, can be one antibody or a combination of two or more antibodies.

The “antibody that specifically recognizes a fusion polypeptide” can be exemplified by an antibody specific to a polypeptide containing the point of fusion of said fusion polypeptide (hereinafter referred to as the “fusion point-specific antibody”). As referred to herein, the “fusion point-specific antibody” means an antibody that specifically binds to the polypeptide containing the point of fusion but does not bind to wild-type proteins from which to derive the segments of the fusion polypeptide each extending toward the N- or C-terminus.

Also, the “antibody that specifically recognizes a fusion polypeptide” can be exemplified by a combination of an antibody binding to a polypeptide consisting of that region of the fusion polypeptide which extends from the point of fusion toward the N-terminus and an antibody binding to a polypeptide consisting of that region of the fusion polypeptide which extends from the point of fusion toward the C-terminus. The fusion polypeptide can be detected by performing sandwich ELISA, immunostaining, immunoprecipitation, Western blotting or the like using these two antibodies.

In the present invention, examples of the antibodies include, but are not limited to, natural antibodies such as polyclonal antibodies and monoclonal antibodies (mAb), and chimeric, humanized and single-stranded antibodies which can be prepared using genetic recombination techniques, and binding fragments thereof. The “binding fragments” refers to partial regions of the above-mentioned antibodies which have specific binding activity, and specific examples include Fab, Fab′, F(ab′)₂, Fv, and single-chain antibodies. The class of antibody is not particularly limited, and any isotype of antibody, such as IgG, IgM, IgA, IgD or IgE, is acceptable, with IgG being preferred in consideration of ease of purification or other factors.

The “antibody that specifically recognizes a fusion polypeptide” can be prepared by those skilled in the art through appropriate selection of a known technique. Examples of such a known technique include: a method in which a polypeptide containing the point of fusion of the fusion polypeptide, a polypeptide consisting of that region of the fusion polypeptide which extends from the point of fusion toward the N-terminus, or a polypeptide consisting of that region of the fusion polypeptide which extends from the point of fusion toward the C-terminus is inoculated into an immune animal, the immune system of the animal is activated, and then the serum (polyclonal antibody) of the animal is collected; as well as monoclonal antibody preparation methods such as hybridoma method, recombinant DNA method, and phage display method. Commercially available antibodies may also be used. If an antibody having a labeling agent attached thereto is used, the target protein can be detected directly by detecting this label. The labeling agent is not particularly limited as long as it is capable of binding to an antibody and is detectable, and examples include peroxidase, β-D-galactosidase, microperoxidase, horseradish peroxidase (HRP), fluorescein isothiocyanate (FITC), rhodamine isothiocyanate (RITC), alkaline phosphatase, biotin, and radioactive materials. Aside from the direct detection of the target protein using the antibody having a labeling agent attached thereto, the target protein can also be detected indirectly using a secondary antibody having a labeling agent attached thereto, Protein G or A, or the like.

<Kit for Detecting Gene Fusions Serving as Responsible Mutations (Driver Mutations) for Cancer>

As described above, fusion polynucleotides produced by gene fusions serving as responsible mutations for cancer, or polypeptides encoded thereby, can be detected using such a primer, probe, or antibody as mentioned above, or a combination thereof, and thereby the gene fusions can be detected. Thus, the present invention provides a kit for detecting a gene fusion serving as a responsible mutation for cancer, the kit comprising any or a combination of (A) to (C) below (hereinafter also referred to as the “inventive kit”):

(A) a polynucleotide that serves as a probe designed to specifically recognize an ATG3-EPHB1 fusion polynucleotide, a TNIK-RNF123 fusion polynucleotide, or an SLC12A2-NRG2 fusion polynucleotide;
(B) polynucleotides that serve as a pair of primers designed to enable specific amplification of an ATG3-EPHB1 fusion polynucleotide, a TNIK-RNF123 fusion polynucleotide, or an SLC12A2-NRG2 fusion polynucleotide; and
(C) an antibody that specifically recognizes an ATG3-EPHB1 fusion polypeptide, a TNIK-RNF123 fusion polypeptide, or an SLC12A2-NRG2 fusion polypeptide.

In addition to the above-mentioned polynucleotide(s) or antibody, the inventive kit can also contain an appropriate combination of other components, including: a substrate required for detecting a label attached to the polynucleotide(s) or the antibody; a positive control (e.g., ATG3-EPHB1 fusion polynucleotide, TNIK-RNF123 fusion polynucleotide, or SLC12A2-NRG2 fusion polynucleotide; or ATG3-EPHB1 fusion polypeptide, TNIK-RNF123 fusion polypeptide, or SLC12A2-NRG2 fusion polypeptide; or cells bearing the same); a negative control; a PCR reagent; a counterstaining reagent for use for in situ hybridization or the like (e.g., DAPI); a molecule required for antibody detection (e.g., secondary antibody, Protein G, Protein A); and a buffer solution for use in sample dilution or washing. The inventive kit can also contain instructions for use thereof. The above-mentioned inventive detection method can be easily carried out by using the inventive kit.

The inventive detection method and kit, which enable detection of the gene fusions newly discovered as responsible mutations for cancer, are very useful in identifying subjects positive for said gene fusion and applying personalized medicine to each of the subjects, as described below.

<Method for Identifying Patients with Cancer or Subjects with a Risk of Cancer>

The above-mentioned three types of gene fusions, serving as responsible mutations for cancer, are each believed to lead to constitutive activation of EPHB1 kinase activity, constitutive activation of TNIK kinase activity, and enhancement of the function of NRG2 as a cell growth factor, thereby contributing to malignant transformation of cancers. Thus, cancer patients in whom such a gene fusion is detected are highly probable to be responsive to the treatment with a substance suppressing the expression and/or activity of a polypeptide encoded by a fusion polynucleotide produced by said gene fusion.

Thus, the present invention provides a method for identifying patients with cancer or subjects with a risk of cancer, in whom substances suppressing the expression and/or activity of polypeptides encoded by fusion polynucleotides produced by gene fusions serving as responsible mutations (driver mutations) for cancer show a therapeutic effect (hereinafter also referred to as the “inventive identification method”).

The inventive identification method comprises the steps of:

(1) detecting a fusion polynucleotide of any one of (a) to (c) below, or a polypeptide encoded thereby, in an isolated sample from a subject:

(a) an ATG3-EPHB1 fusion polynucleotide which encodes a polypeptide comprising the kinase domain of EPHB1 and having kinase activity;

(b) a TNIK-RNF123 fusion polynucleotide which encodes a polypeptide comprising the kinase domain of TNIK and the SPRY domain and RING finger domain of RNF123, and having kinase activity; and

(c) an SLC12A2-NRG2 fusion polynucleotide which encodes a polypeptide comprising the AA_permease_N super family domain of SLC12A2 and the neuregulin family conserved region of NRG2, and having intracellular signaling-enhancing activity; and

(2) determining that the substance suppressing the expression and/or activity of the polypeptide shows a therapeutic effect in the subject, in the case where the fusion polynucleotide of any one of (a) to (c) or the polypeptide encoded thereby is detected.

In the inventive identification method, the phrase “a patient with cancer or a subject with a risk of cancer” refers to a mammal, preferably human, which is, or is suspected of being, affected by cancer. The “cancer” to which the inventive identification method is to be applied is not particularly limited as long as it is a cancer in which any of the above-mentioned three types of gene fusions can be detected, with gastric cancer being preferred, and special-type gastric cancer, undifferentiated gastric cancer, or moderately differentiated gastric cancer being more preferred.

In the inventive identification method, the “therapeutic effect” is not particularly limited as long as it is a beneficial effect of cancer treatment for a patient, and examples include a tumor shrinkage effect, a progression-free survival prolongation effect, and a life lengthening effect.

In the inventive identification method, the “substance suppressing the expression and/or activity of a polypeptide encoded by a fusion polynucleotide produced by a gene fusion serving as a responsible mutation (driver mutation) for cancer”, which is to be evaluated for its effectiveness in the treatment of cancer associated with the ATG3-EPHB1 gene fusion (this substance is hereinafter also referred to as the “substance to be evaluated in the inventive identification method”), is not particularly limited as long as it is a substance that directly or indirectly inhibits the expression and/or function of an ATG3-EPHB1 fusion polypeptide.

Examples of the substance inhibiting the expression of an ATG3-EPHB1 fusion polypeptide include: siRNAs (small interfering RNAs), shRNAs (short hairpin RNAs), miRNAs (micro RNAs), and antisense nucleic acids which suppress the expression of an ATG3-EPHB1 fusion polypeptide; expression vectors capable of expressing these polynucleotides; and low-molecular-weight compounds.

Examples of the substance inhibiting the function of an ATG3-EPHB1 fusion polypeptide include substances inhibiting EPHB1 kinase activity (e.g., low-molecular-weight compounds), and antibodies binding to an ATG3-EPHB1 fusion polypeptide.

These substances may be substances that specifically suppress the expression and/or activity of an ATG3-EPHB1 fusion polypeptide, or may be substances that suppress even the expression and/or activity of wild-type EPHB1 protein. Specific examples of such substances include, but are not limited to, dasatinib, PD-173955, foretinib, bosutinib, crizotinib, AST-487, staurosporine, vandetanib, MLN-8054, TAE 684, nilotinib, dorsomorphin, Cdk1/2 inhibitor III, IKK-2 inhibitor IV, GSK-3β inhibitor XII, and PP1 Analog II (cf., Ephrin receptor family: EPH receptor B1. Last modified on 26 Mar. 2013. Accessed on Oct. 12, 2013. IUPHAR database (IUPHAR-DB), http://www.iuphar-db.org/DATABASE/ObjectDisplayForward?objectId=1830).

These substances can be prepared by a per se known technique on the basis of the sequence information of an ATG3-EPHB1 fusion polynucleotide and/or an ATG3-EPHB1 fusion polypeptide which are disclosed in the present specification, or other data. Commercially available substances may also be used.

These substances are effective as cancer therapeutic agents for a subject, in the case where an ATG3-EPHB1 fusion polynucleotide or a polypeptide encoded thereby is detected in an isolated sample from said subject.

As regards TNIK-RNF123 gene fusion, the substance to be evaluated in the inventive identification method is not particularly limited as long as it is a substance that directly or indirectly inhibits the expression and/or function of a TNIK-RNF123 fusion polypeptide.

Examples of the substance inhibiting the expression of a TNIK-RNF123 fusion polypeptide include: siRNAs (small interfering RNAs), shRNAs (short hairpin RNAs), miRNAs (micro RNAs), and antisense nucleic acids which suppress the expression of a TNIK-RNF123 fusion polypeptide; expression vectors capable of expressing these polynucleotides; and low-molecular-weight compounds.

Examples of the substance inhibiting the function of a TNIK-RNF123 fusion polypeptide include substances inhibiting TNIK kinase activity (e.g., low-molecular-weight compounds), and antibodies binding to a TNIK-RNF123 fusion polypeptide.

These substances may be substances that specifically suppress the expression and/or activity of a TNIK-RNF123 fusion polypeptide, or may be substances that suppress even the expression and/or activity of wild-type TNIK protein. Specific examples of such substances include, but are not limited to, aminothiazole derivatives (refer to WO 2010/064111) and 4-phenyl-2-phenylaminopyridine-based compounds (refer to Ho K. K., et al., Bioorg Med Chem Lett, 2013, 23 (2): 569-73 (Epub 16 Nov. 2012)).

These substances can be prepared by a per se known technique on the basis of the sequence information of a TNIK-RNF123 fusion polynucleotide and/or a TNIK-RNF123 fusion polypeptide which are disclosed in the present specification, or other data. Commercially available substances may also be used.

These substances are effective as cancer therapeutic agents for a subject, in the case where a TNIK-RNF123 fusion polynucleotide or a polypeptide encoded thereby is detected in an isolated sample from said subject.

As regards SLC12A2-NRG2 gene fusion, the substance to be evaluated in the inventive identification method is not particularly limited as long as it is a substance that directly or indirectly inhibits the expression and/or function of an SLC12A2-NRG2 fusion polypeptide.

Examples of the substance inhibiting the expression of an SLC12A2-NRG2 fusion polypeptide include: siRNAs (small interfering RNAs), shRNAs (short hairpin RNAs), miRNAs (micro RNAs), and antisense nucleic acids which suppress the expression of an SLC12A2-NRG2 fusion polypeptide; expression vectors capable of expressing these polynucleotides; and low-molecular-weight compounds.

Examples of the substance inhibiting the function of an SLC12A2-NRG2 fusion polypeptide include substances inhibiting the intracellular signaling-enhancing activity of NRG2 (e.g., low-molecular-weight compounds), and antibodies binding to an SLC12A2-NRG2 fusion polypeptide.

These substances may be substances that specifically suppress the expression and/or activity of an SLC12A2-NRG2 fusion polypeptide, or may be substances that suppress even the expression and/or activity of wild-type NRG2 protein.

These substances can be prepared by a per se known technique on the basis of the sequence information of an SLC12A2-NRG2 fusion polynucleotide and/or an SLC12A2-NRG2 fusion polypeptide which are disclosed in the present specification, or other data. Commercially available substances may also be used.

Further, since wild-type NRG2 protein is believed to enhance intracellular signaling via a protein belonging to a group of HER proteins serving as receptors for the wild-type NRG2 protein, the substance to be evaluated in the inventive identification method can also be exemplified by substances that directly or indirectly inhibit the expression and/or function of HER proteins. As referred to herein, the group of HER proteins is a group of tyrosine kinase receptors which consists of the following four proteins: HER1 (ErbB1), HER2 (ErbB2), HER3 (ErbB3), and HER4 (ErbB4).

Examples of the substances inhibiting the expression of HER proteins include: siRNAs (small interfering RNAs), shRNAs (short hairpin RNAs), miRNAs (micro RNAs), and antisense nucleic acids which suppress the expression of HER proteins; expression vectors capable of expressing these polynucleotides; and low-molecular-weight compounds.

Examples of the substances inhibiting the function of HER proteins include substances inhibiting the kinase activity of HER proteins (e.g., low-molecular-weight compounds), and antibodies binding to HER proteins. Specific examples of such substances include rapatinib, afatinib, dacomitinib, and trastuzumab.

These substances can be prepared by a per se known technique on the basis of known sequence information or other data. Commercially available substances may also be used.

These substances are effective as cancer therapeutic agents for a subject, in the case where an SLC12A2-NRG2 fusion polynucleotide or a polypeptide encoded thereby is detected in an isolated sample from said subject.

Step (1) in the inventive identification method can be carried out in the same way as the step included in the inventive detection method mentioned above.

At step (2) in the inventive identification method, the substance to be evaluated in the inventive identification method is determined to show a therapeutic effect in a subject (i.e., a patient with cancer or a subject with a risk of cancer), in the case where a fusion polynucleotide of interest or a polypeptide encoded thereby is detected in an isolated sample from the subject at step (1); however, the substance to be evaluated in the inventive identification method is determined to be unlikely to show a therapeutic effect in the subject, in the case where neither the fusion polynucleotide of interest nor the polypeptide encoded thereby is detected.

According to the inventive identification method, it is possible to detect subjects positive for the gene fusions newly discovered as responsible mutations for cancer from among patients with cancer or subjects with a risk of cancer, and to identify patients with cancer or subjects with a risk of cancer, in whom substances suppressing the expression and/or activity of polypeptides encoded by fusion polynucleotides produced by said gene fusions show a therapeutic effect; thus, the present invention is useful in that it enables provision of suitable treatment for such subjects.

<Method for Treatment of Cancer and Cancer Therapeutic Agent>

As described above, the inventive identification method allows identification of patients with cancer, in whom substances suppressing the expression and/or activity of polypeptides encoded by fusion polynucleotides produced by any of the above-mentioned three types of gene fusions show a therapeutic effect. Thus, efficient cancer treatment can be performed by administering said substance selectively to a cancer patient who carries said fusion gene. Therefore, the present invention provides a method for treating cancer, comprising the step of administering said substance to a subject (hereinafter also referred to as the “inventive treatment method”).

In the inventive treatment method, the subject is typified by a patient with cancer positive for ATG3-EPHB1, TNIK-RNF123 or SLC12A2-NRG2 gene fusion. The cancer is not particularly limited, and is preferably gastric cancer, more preferably special-type gastric cancer, undifferentiated gastric cancer, or moderately differentiated gastric cancer. As referred to herein, the cancer positive for ATG3-EPHB1, TNIK-RNF123 or SLC12A2-NRG2 gene fusion refers to a cancer in which a fusion polynucleotide or a polypeptide encoded thereby is detected, for any of these gene fusions, by the inventive detection method.

Also, the present invention provides a therapeutic agent for cancer positive for ATG3-EPHB1, TNIK-RNF123 or SLC12A2-NRG2 gene fusion (hereinafter also referred to as the “inventive cancer therapeutic agent”). For the cancers positive for the above-mentioned three types of gene fusions, no treatment targeting these gene fusions has been performed. Also, the presence of these gene fusions was totally unpredictable even in consideration of any prior art documents or other information. Therefore, the inventive cancer therapeutic agent provides novel therapeutic measures effective for the cancers positive for the above-mentioned three types of gene fusions.

In the inventive cancer therapeutic agent, the cancer is not particularly limited, and is preferably gastric cancer, more preferably special-type gastric cancer, undifferentiated gastric cancer, or moderately differentiated gastric cancer.

The inventive cancer therapeutic agent typically comprises, as an active ingredient, a substance suppressing the expression and/or activity of: an ATG3-EPHB1 fusion polypeptide comprising the kinase domain of EPHB1 and having kinase activity; a TNIK-RNF123 fusion polypeptide comprising the kinase domain of TNIK and the SPRY domain and RING finger domain of RNF123 and having kinase activity; or an SLC12A2-NRG2 fusion polypeptide comprising the AA_permease_N super family domain of SLC12A2 and the neuregulin family conserved region of NRG2, and having intracellular signaling-enhancing activity.

In the context of the inventive treatment method and inventive cancer therapeutic agent, the substances suppressing the expression and/or activity of the above-mentioned three types of fusion polypeptides can be exemplified by the substances listed above in <Method for identifying patients with cancer or subjects with a risk of cancer>.

The inventive cancer therapeutic agent can be formulated into a pharmaceutical composition with the use of a pharmaceutically acceptable carrier, excipient and/or other additives which are commonly used in pharmaceutical manufacturing.

The method for administering the inventive cancer therapeutic agent is selected as appropriate depending on various factors including the type of this suppressing agent and the type of cancer, and exemplary modes of administration that can be adopted include oral, intravenous, intraperitoneal, transdermal, intramuscular, intratracheal (aerosol), rectal and intravaginal administrations.

The dose of the inventive cancer therapeutic agent can be determined as appropriate in consideration of various factors, including the activity and type of an active ingredient, the mode of administration (e.g., oral or parenteral administration), the severity of a disease, and the animal species, drug receptivity, body weight, and age of the subject to be treated with the inventive agent.

The treatment method and cancer therapeutic agent of the present invention are useful in that they allow treatment of patients having the particular responsible mutations for cancer which have been conventionally unknown and were first discovered according to this invention.

<Method for Screening Cancer Therapeutic Agents>

The present invention provides a method for screening cancer therapeutic agents which show a therapeutic effect in cancer patients having any of the above-mentioned three types of gene fusions (hereinafter also referred to as the “inventive screening method”). According to the inventive screening method, substances suppressing the expression and/or activity of any of the above-mentioned three types of fusion polypeptides (i.e., ATG3-EPHB1 fusion polypeptide, TNIK-RNF123 fusion polypeptide, and SLC12A2-NRG2 fusion polypeptide) can be obtained as cancer therapeutic agents.

The test substance to be subjected to the inventive screening method can be any compound or composition, and can be exemplified by nucleic acids (e.g., nucleoside, oligonucleotide, polynucleotide), saccharides (e.g., monosaccharide, disaccharide, oligosaccharide, polysaccharide), fats (e.g., saturated or unsaturated, straight-chain, branched-chain and/or cyclic fatty acids), amino acids, proteins (e.g., oligopeptide, polypeptide), low-molecular-weight compounds, compound libraries, random peptide libraries, natural ingredients (e.g., ingredients derived from microbes, animals and plants, marine organisms, and others), foods, and the like.

The inventive screening method can be of any type as long as it enables evaluation of whether a test substance suppresses the expression and/or activity of any of the above-mentioned three types of fusion polypeptides. Typically, the inventive screening method comprises the following steps:

(1) bringing a cell expressing an ATG3-EPHB1 fusion polypeptide, a TNIK-RNF123 fusion polypeptide, or an SLC12A2-NRG2 fusion polypeptide into contact with a test substance;
(2) determining whether the expression and/or activity of any of said fusion polypeptides is suppressed or not; and
(3) selecting the substance determined to suppress the expression and/or activity of any of said fusion polypeptides, as a cancer therapeutic agent.

At step (1), the cell expressing any of the above-mentioned three types of fusion polypeptides is brought into contact with the test substance. A test substance-free solvent (e.g., DMSO) can be used as a control. The contact can be effected in a medium. The medium is selected as appropriate depending on various factors including the type of the cell to be used, and examples include a minimum essential medium (MEM) supplemented with about 5-20% fetal bovine serum, a Dulbecco's modified eagle's medium (DMEM), RPMI1640 medium, and 199 medium. The culture conditions are also selected as appropriate depending on various factors including the type of the cell to be used, and for example, the pH of the medium is in the range of approximately from 6 to 8, the culture temperature is in the range of approximately from 30 to 40° C., and the culture time is in the range of approximately from 12 to 72 hours.

Examples of the cell expressing any of the above-mentioned three types of fusion polypeptides include, but are not limited to, cancer tissue-derived cells intrinsically expressing said fusion polypeptides, cell lines induced from said cells, and cell lines made by genetic engineering. Whether a cell expresses any of the above-mentioned three types of fusion polypeptides can also be confirmed using the inventive detection method described above. The cell is generally a mammalian cell, preferably a human cell.

At step (2), it is determined whether the test substance suppresses the expression and/or activity of any of said fusion polypeptides or not. The expression of fusion polypeptides can be measured by determining the mRNA or protein level in a cell using a known analysis technique such as Northern blotting, quantitative PCR, immunoblotting, or ELISA. Also, the activity of fusion polypeptides can be measured by a known analysis technique (e.g., kinase activity assay). A resulting measured value is compared with a value measured in a control cell not contacted with the test substance. The comparison of the measured values is made preferably based on the presence or absence of a significant difference. If the value measured in the cell contacted with the test substance is significantly lower than that measured in the control cell, it can be determined that the test substance suppresses the expression and/or activity of any of said fusion polypeptides.

Alternatively, given the fact that the cells expressing these types of fusion polypeptides show enhanced growth, the growth of said cells can be used as an indicator for the determination at this step. In this case, the growth of such a cell contacted with the test substance is measured as a first step. The cell growth measurement can be made by a per se known technique such as cell count, ³H-thymidine incorporation, or BRDU. Next, the growth of the cell contacted with the test substance is compared with that of a control cell not contacted with the test substance. The growth level comparison is made preferably based on the presence or absence of a significant difference. The value for the growth of the control cell not contacted with the test substance can be measured prior to, or at the same time as, the measurement of the growth of the cell contacted with the test substance, but is preferably measured at the same time from the viewpoints of the accuracy and reproducibility of the test. If the results of the comparison show that the growth of the cell contacted with the test substance is suppressed, it can be determined that the test substance suppresses the expression and/or activity of any of said fusion polypeptides.

At step (3), the test substance determined to suppress the expression and/or activity of any of said fusion polypeptides at step (2) is selected as a cancer therapeutic agent.

Hence, the inventive screening method makes it possible to obtain cancer therapeutic agents applicable to the treatment of patients with responsible mutations for cancer which have been conventionally unknown.

<Isolated Fusion Polypeptides or Fragments Thereof, and Polynucleotides Encoding the Same>

The present invention provides isolated fusion polypeptides (hereinafter also referred to as the “inventive fusion polypeptides”) as mentioned below, or fragments thereof:

(1) an isolated ATG3-EPHB1 fusion polypeptide which comprises the kinase domain of EPHB1 protein and has kinase activity;
(2) an isolated TNIK-RNF123 fusion polypeptide which comprises the kinase domain of TNIK protein and the SPRY domain and RING finger domain of RNF123 protein and has kinase activity; and
(3) an isolated SLC12A2-NRG2 fusion polypeptide which comprises the AA_permease_N super family domain of SLC12A2 protein and the neuregulin family conserved region of NRG2 protein, and has intracellular signaling-enhancing activity.

For the purpose of the present specification, the “isolated” substance refers to a substance substantially separated or purified from other substances (preferably, biological factors) found in an environment in which the substance naturally occurs (e.g., in a cell of an organism) (for example, if the substance of interest is a nucleic acid, the “other substances” corresponds to other factors than nucleic acids as well as nucleic acids containing other nucleic acid sequences than that of the nucleic acid of interest; and if the substance of interest is a protein, the “other substances” corresponds to other factors than proteins as well as proteins containing other amino acid sequences than that of the protein of interest). For the purpose of the specification, the term “isolated” means that a substance has a purity of preferably at least 75% by weight, more preferably at least 85% by weight, still more preferably at least 95% by weight, and most preferably at least 96% by weight, at least 97% by weight, at least 98% by weight, at least 99% by weight, or 100%. Examples of the “isolated” polynucleotide and polypeptide include not only polynucleotides and polypeptides purified by standard purification techniques but also chemically synthesized polynucleotides and polypeptides.

Other terms used above in (1) to (3) have the same meanings as defined above in <Specific responsible mutations for cancer>.

The “fragments” refers to fragments of the inventive fusion polypeptides, which each consist of a consecutive partial sequence comprising sequences upstream and downstream from the point of fusion. The sequence upstream from the point of fusion, as contained in said partial sequence, can comprise at least one amino acid residue (for example, at least 2, 3, 4, 5, 6, 7, 8, 9, 10, 20, 30, 40, 50 or 100 amino acid residues) ranging from the point of fusion to the N-terminus of any of the inventive fusion polypeptides. The sequence downstream from the point of fusion, as contained in said partial sequence, can comprise at least one amino acid residue (for example, at least 2, 3, 4, 5, 6, 7, 8, 9, 10, 20, 30, 40, 50 or 100 amino acid residues) ranging from the point of fusion to the C-terminus of any of the inventive fusion polypeptides. The length of the fragments is not particularly limited, and is generally at least 8 amino acid residues (for example, at least 9, 10, 11, 12, 13, 14, 15, 20, 25, 50 or 100 amino acid residues).

Also, the inventive fusion polypeptides can be, for example, the isolated fusion polypeptides mentioned below:

(1) an ATG3-EPHB1 fusion polypeptide which is any one of (i) to (iii) below:

(i) a polypeptide consisting of the amino acid sequence of SEQ ID NO: 2,

(ii) a polypeptide consisting of an amino acid sequence derived from the amino acid sequence of SEQ ID NO: 2 by deletion, substitution or addition of one or more amino acids, and the polypeptide having kinase activity, or

(iii) a polypeptide consisting of an amino acid sequence having a sequence identity of at least 80% to the amino acid sequence of SEQ ID NO: 2, and the polypeptide having kinase activity;

(2) a TNIK-RNF123 fusion polypeptide which is any one of (i) to (iii) below:

(i) a polypeptide consisting of the amino acid sequence of SEQ ID NO: 4, 6, 8, 10, 12, 14, 16 or 18,

(ii) a polypeptide consisting of an amino acid sequence derived from the amino acid sequence of SEQ ID NO: 4, 6, 8, 10, 12, 14, 16 or 18 by deletion, substitution or addition of one or more amino acids, and the polypeptide having kinase activity, or

(iii) a polypeptide consisting of an amino acid sequence having a sequence identity of at least 80% to the amino acid sequence of SEQ ID NO: 4, 6, 8, 10, 12, 14, 16 or 18, and the polypeptide having kinase activity; and

(3) an SLC12A2-NRG2 fusion polypeptide which is any one of (i) to (iii) below:

(i) a polypeptide consisting of the amino acid sequence of SEQ ID NO: 20, 22, 24, 26 or 28,

(ii) a polypeptide consisting of an amino acid sequence derived from the amino acid sequence of SEQ ID NO: 20, 22, 24, 26 or 28 by deletion, substitution or addition of one or more amino acids, and the polypeptide having intracellular signaling-enhancing activity, or

(iii) a polypeptide consisting of an amino acid sequence having a sequence identity of at least 80% to the amino acid sequence of SEQ ID NO: 20, 22, 24, 26 or 28, and the polypeptide having intracellular signaling-enhancing activity.

The terms used above in (i) to (iii) have the same meanings as defined above in <Specific responsible mutations for cancer>.

Further, the present invention provides isolated polynucleotides encoding the inventive fusion polypeptides or the fragments thereof as described above (hereinafter also referred to as the “inventive polynucleotides”). The inventive polynucleotides can be in the form of any of mRNA, cDNA and genomic DNA. Also, the polynucleotides may be double- or single-stranded.

A typical example of the cDNA encoding the ATG3-EPHB1 fusion polypeptide is a polynucleotide consisting of the nucleotide sequence of SEQ ID NO: 1.

Typical examples of the cDNA encoding the TNIK-RNF123 fusion polypeptide are polynucleotides each consisting of the nucleotide sequence of SEQ ID NO: 3, 5, 7, 9, 11, 13, 15 or 17.

Typical examples of the cDNA encoding the SLC12A2-NRG2 fusion polypeptide are polynucleotides each consisting of the nucleotide sequence of SEQ ID NO: 19, 21, 23, 25 or 27.

The inventive polynucleotides can be made by a per se known technique. For example, the inventive polynucleotides can be extracted using a known hybridization technique from a cDNA library or genomic library prepared from cancer tissues or the like harboring an ATG3-EPHB1 fusion polynucleotide, a TNIK-RNF123 fusion polynucleotide, or an SLC12A2-NRG2 fusion polynucleotide. The inventive polynucleotides can also be prepared by amplification utilizing a known gene amplification technique (PCR), with mRNA, cDNA or genomic DNA prepared from the cancer tissues or the like being used as a template. Alternatively, the polynucleotides can be prepared utilizing a known gene amplification or genetic recombination technique such as PCR, restriction enzyme treatment, or site-directed mutagenesis (Kramer, W. & Fritz, H. J., Methods Enzymol., 1987, 154, 350), using, as starting materials, the cDNAs of wild-type genes from which to derive those segments of each of the fusion polynucleotides which extend toward the 5′- or 3′-end.

The inventive fusion polypeptides or fragments thereof can also be made by a per se known technique. For example, after such a polynucleotide prepared as mentioned above is inserted into an appropriate expression vector, the vector is introduced into a cell-free protein synthesis system (e.g., reticulocyte extract, wheat germ extract) and the system is incubated, or alternatively the vector is introduced into appropriate cells (e.g., E coli., yeast, insect cells, animal cells) and the resulting transformant is cultured; in either of the above ways, the inventive fusion polypeptides can be prepared.

The inventive fusion polypeptides or fragments thereof can be used as a marker in the inventive detection method or the like, or can be used in other applications including preparation of antibodies against the inventive fusion polypeptides.

### EXAMPLES

Hereunder, the present invention will be more specifically described by way of working examples, but this invention is not limited to the examples given below.

<Samples>

As samples for RNA sequencing, total RNAs were prepared by a conventional method from gastric tissues taken from 51 patients with gastric cancer (GC) (5 with moderately differentiated GC, 3 with special-type mucinous GC, and 43 with undifferentiated GC). As samples for RT-PCR and Sanger sequencing, total RNAs were prepared by a conventional method from gastric tissues taken from 149 patients with gastric cancer (27 with highly differentiated GC, 65 with moderately differentiated GC, 2 with special-type mucinous GC, and 55 with undifferentiated GC). The present study was conducted with the approval by the institutional review boards of the institutions involved in the study.

<RNA Sequencing>

RNA sequencing libraries were prepared using the TruSeq RNA Sample Prep Kit v2 produced by Illumina according to the manufacturer's standard protocol. These libraries were subjected to paired-end sequencing of 100 by reads on the next-generation sequencer Hiseq2000 or GAIIx. The resulting paired-end reads were compared to the RefSeq (NCBI Reference Sequence) database to thereby detect fusion gene candidates.

<RT-PCR, Sanger Sequencing>

cDNAs were synthesized by a reverse transcriptase method from the total RNAs extracted from the gastric tissues taken from the gastric cancer patients. The resulting cDNAs were subjected to PCR amplification. The obtained PCR products were directly nucleotide-sequenced using the BigDye Terminator Kit and the ABI 3130xl DNA Sequencer (Applied Biosystems). The primers used in this study are shown in Table 1.

TABLE 1

| | | | RT-PCR |
|---|---|---|---|
| | | | product |
| Fusion | | Anti-sense | size |
| gene | Sense primer | primer | (bp) |
| ATG3- | CACTGGAAGTGGCTG | GCCGCAGCCCATCAA | 150 |
| EPHB1 | AGTACCTGA | TCCTT | |
| | (SEQ ID NO: 65) | (SEQ ID NO: 66) | |
| TNIK- | GGTCAGTGGAAACAG | ACGCCTTGCCATAAT | 318 |
| RNF123 | GACATTTGG | TCGTTGT | |
| | (SEQ ID NO: 67) | (SEQ ID NO: 68) | |
| SLC12A2- | GGTCAAGCTGGAATA | TGCCTCACACTTCAG | 189 |
| NRG2 | GGTCTATCAG | CGATT | |
| | (SEQ ID NO: 39) | (SEQ ID NO: 70) | |

### Example

This example describes the identification of novel fusion transcripts in gastric tissues.

In order to identify novel fusion transcripts potentially serving as targets for treatment, the gastric tissues taken from 51 patients with gastric cancer (5 with moderately differentiated GC, 3 with special-type mucinous GC, and 43 with undifferentiated GC) were subjected to whole-transcriptome sequencing (RNA sequencing; refer to Meyerson, M., et al., Nat. Rev. Genet., 2010, 11, 685-696).

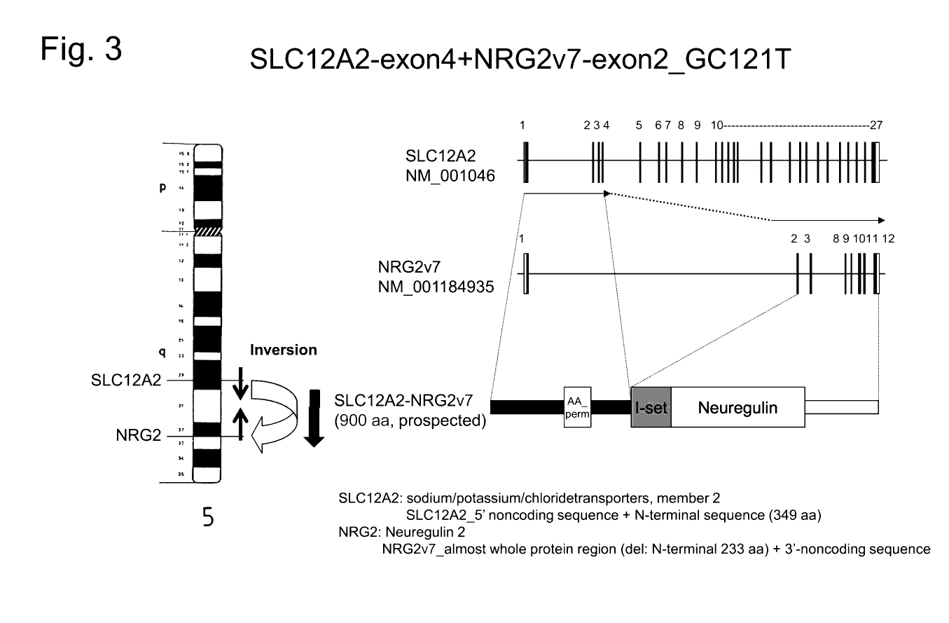

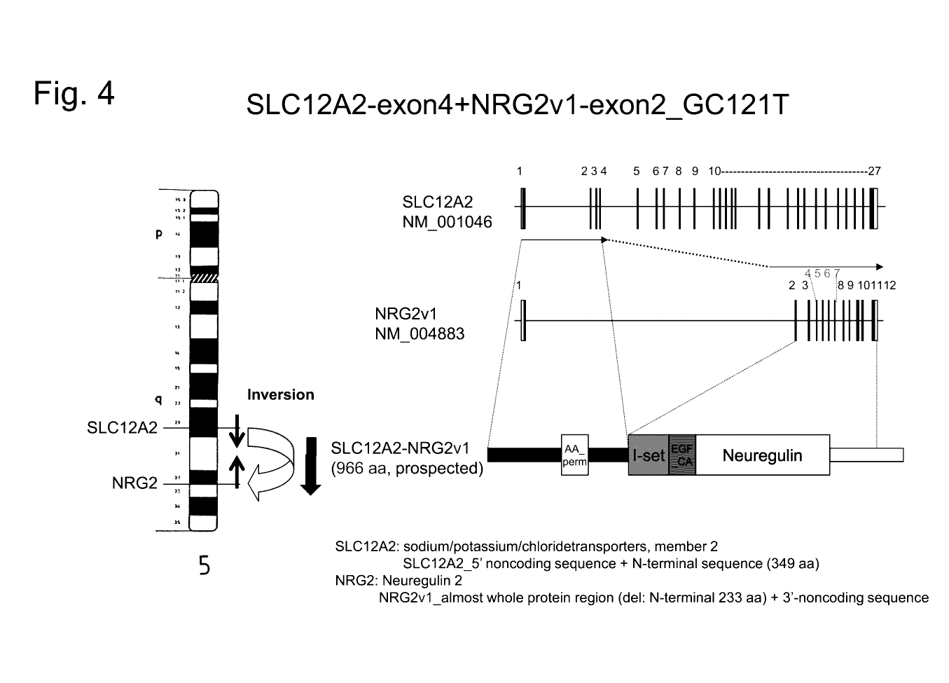

After overlapping clones generated by PCR were eliminated, at least 8.0×10⁷ paired nucleotide sequences were obtained for each case. These sequences were compared to existing gene databases; as a result, a candidate for fusion gene between the ATG3 gene and the EPHB1 gene was detected in one case (special-type mucinous GC patient), a candidate for fusion gene between the TNIK gene and the RNF123 gene was detected in one case (undifferentiated GC patient), and a candidate for fusion gene between the SLC12A2 gene and the NRG2 gene was detected in one case (moderately differentiated GC patient). The structures of these fusion genes are shown in Table 2 and FIGS. 1 to 4.

TABLE 2

| | Location | Location | Causative |
|---|---|---|---|
| | of gene | of gene | chromosomal |
| Fusion gene | toward 5′-end | toward 3′-end | aberration |
| ATG3-EPHB1 | 3q13.2 | 3q21-q23 | Chromosomal |
| | | | inversion (inv3) |
| TNIK-RNF123 | 3q26.31 | 3p24.3 | Chromosomal |
| | | | inversion (inv3) |
| SLC12A2-NRG2 | 5q23.3 | 5q23-q33 | Chromosomal |
| | | | inversion (inv5) |

Then, RT-PCR was performed using the primers shown in Table 1 on the specimens of the cases in which fusion genes were detected, and the obtained PCR products were subjected to sequence analysis to verify the expression of the above-mentioned three types of fusion genes. The sequences of the amplified PCR products are as follows. ATG3-EPHB1 (Uppercase and lowercase letters represent an ATG3 gene-derived sequence and an EPHB1 gene-derived sequence, respectively):

(SEQ ID NO: 71)
CACTGGAAGTGGCTGAGTACCTGACCCCGGTCCTCAAGGAATCAAAGTTT
AAGGAAACAGGTGTAATTACCCCAGAAGAGgaacacaatgagttcaactc
ctccatggccaggagtcagaccaacacagcaaggattgatgggctgcggc

TNIK-RNF123 (Uppercase and lowercase letters represent a TNIK gene-derived sequence and an RNF123 gene-derived sequence, respectively):

(SEQ ID NO: 72)
GGTCAGTGGAAACAGGACATTTGGATGGAGTATTTATGCATAAGCGAGCT
CAAAGGTTAAAGTTTCTATGTGAAAGAAATGATAAGgtgattggacacag
caactttggcaccatccgctctaccacatgcgtgtacaaagggaaatggc
tctacgaggtcctcatctcctcccaggggctcatgcagatcggctggtgc
accatcagctgccgcttcaaccaggaggagggggttggagatacacacaa
ctcctatgcctatgatggcaaccgcgtgcgcaagtggaatgtgaccacaa
cgaattatggcaaggcgt

SLC12A2-NRG2 fusion gene (Uppercase and lowercase letters represent an SLC12A2 gene-derived sequence and an NRG2 gene-derived sequence, respectively):

(SEQ ID NO: 73)
GGTCAAGCTGGAATAGGTCTATCAGTCCTTGTAATAATGATGGCCACTGT
TGTGACAACTATCACAGGATTGTCTACTTCAGCAATAGCAACTAATGGAT
TTGTAAGAGGAGccacccggcccaagttgaagaagatgaagagccagacg
ggacaggtgggtgagaagcaatcgctgaagtgtgaggca

ATG3-EPHB1 is a fusion gene generated by chromosomal inversion (inv3), in which exon 2 of the ATG3 gene is directly bound to exon 7 of the EPHB1 gene without inconsistency in the reading frames of these genes. The entire coding region of the ATG3-EPHB1 fusion gene was cloned and sequenced (SEQ ID NO: 1).

TNIK-RNF123 is a fusion gene generated by chromosomal inversion (inv3), in which exon 32 of the TNIK gene is directly bound to exon 6 of the RNF123 gene without inconsistency in the reading frames of these genes.

SLC12A2-NRG2 is a fusion gene generated by chromosomal inversion (inv5), in which exon 4 of the SLC12A2 gene is directly bound to exon 2 of the NRG2 gene without inconsistency in the reading frames of these genes.

The above results demonstrated that the gene fusions found in Example can be detected by verifying the presence or absence of the amplification of specific bands by RT-PCR and sequencing the nucleotide sequences of the PCR products.

Further, RT-PCR was performed on normal gastric mucosal tissues using the primers shown in Table 1, and as a result, no amplification of a specific band was observed, which demonstrated that none of the above-mentioned three types of fusion genes was expressed. The results indicated that the above-mentioned three types of gene fusions were specific for cancer cells.

Furthermore, none of KRAS mutations (mutations at the 12th, 13rd or 61st amino acid residue from the N-terminus), NRAS mutations (mutations at the 12th, 13rd or 61st amino acid residue from the N-terminus), and BRAF mutations (mutations at the 600th amino acid residue from the N-terminus and its neighbors), which were known as causative gene mutations for gastric cancer, was detected in any of the three cases in which any of the above-mentioned three types of fusion genes was detected. Therefore, it was suggested that these fusion genes are highly likely to be responsible mutations for gastric cancer.

### INDUSTRIAL APPLICABILITY

The present invention makes it possible to detect gene fusions newly discovered as responsible mutations for cancer; to identify patients with cancer or subjects with a risk of cancer, in whom substances suppressing the expression and/or activity of polypeptides encoded by fusion polynucleotides produced by said gene fusions show a therapeutic effect; and to provide suitable treatment (personalized medicine) for such cancer patients.

### Sequence Listing Free Text

SEQ ID NO: 1: ATG3-EPHB1 fusion polynucleotide
SEQ ID NO: 2: ATG3-EPHB1 fusion polypeptide
SEQ ID NOs: 3, 5, 7, 9, 11, 13, 15 and 17: TNIK-RNF123 fusion polynucleotides
SEQ ID NOs: 4, 6, 8, 10, 12, 14, 16 and 18: TNIK-RNF123 fusion polypeptides
SEQ ID NOs: 19, 21, 23, 25 and 27: SLC12A2-NRG2 fusion polynucleotides
SEQ ID NOs: 20, 22, 24, 26 and 28: SLC12A2-NRG2 fusion polypeptides
SEQ ID NO: 29: ATG3 cDNA
SEQ ID NO: 30: ATG3 polypeptide
SEQ ID NO: 31: EPHB1 cDNA
SEQ ID NO: 32: EPHB1 polypeptide
SEQ ID NOs: 33, 35, 37, 39, 41, 43, 45 and 47: TNIK cDNAs
SEQ ID NOs: 34, 36, 38, 40, 42, 44, 46 and 48: TNIK polypeptides
SEQ ID NO: 49: RNF123 cDNA
SEQ ID NO: 50: RNF123 polypeptide
SEQ ID NOs: 51 and 53: SLC12A2 cDNAs
SEQ ID NOs: 52 and 54: SLC12A2 polypeptides
SEQ ID NOs: 55, 57, 59, 61 and 63: NRG2 cDNAs
SEQ ID NOs: 56, 58, 60, 62 and 64: NRG2 polypeptides
SEQ ID NO: 65: ATG3 sense primer
SEQ ID NO: 66: EPHB1 primer
SEQ ID NO: 67: TNIK sense primer
SEQ ID NO: 68: RNF123 primer
SEQ ID NO: 69: SLC12A2 sense primer
SEQ ID NO: 70: NRG2 primer
SEQ ID NO: 71: Partial sequence of ATG3-EPHB1 fusion polynucleotide
SEQ ID NO: 72: Partial sequence of TNIK-RNF123 fusion polynucleotide
SEQ ID NO: 73: Partial sequence of SLC12A2-NRG2 fusion polynucleotide

1. A method for detecting a gene fusion serving as a responsible mutation (driver mutation) for cancer, the method comprising the step of detecting a fusion polynucleotide of any one of (a) to (c) below, or a polypeptide encoded thereby, in an isolated sample from a subject:
(a) an ATG3-EPHB1 fusion polynucleotide which encodes a polypeptide comprising the kinase domain of EPHB1 and having kinase activity;
(b) a TNIK-RNF123 fusion polynucleotide which encodes a polypeptide comprising the kinase domain of TNIK and the SPRY domain and RING finger domain of RNF123, and having kinase activity; and
(c) an SLC12A2-NRG2 fusion polynucleotide which encodes a polypeptide comprising the AA_permease_N super family domain of SLC12A2 and the neuregulin family conserved region of NRG2, and having intracellular signaling-enhancing activity.

2. The method according to claim 1, wherein the fusion polynucleotide is any one of (a) to (c) below:
(a) an ATG3-EPHB1 fusion polynucleotide encoding the polypeptide mentioned below:
(i) a polypeptide consisting of the amino acid sequence of SEQ ID NO: 2,
(ii) a polypeptide consisting of an amino acid sequence derived from the amino acid sequence of SEQ ID NO: 2 by deletion, substitution or addition of one or more amino acids, and the polypeptide having kinase activity, or
(iii) a polypeptide consisting of an amino acid sequence having a sequence identity of at least 80% to the amino acid sequence of SEQ ID NO: 2, and the polypeptide having kinase activity;
(b) a TNIK-RNF123 fusion polynucleotide encoding the polypeptide mentioned below:
(i) a polypeptide consisting of the amino acid sequence of SEQ ID NO: 4, 6, 8, 10, 12, 14, 16 or 18,
(ii) a polypeptide consisting of an amino acid sequence derived from the amino acid sequence of SEQ ID NO: 4, 6, 8, 10, 12, 14, 16 or 18 by deletion, substitution or addition of one or more amino acids, and the polypeptide having kinase activity, or
(iii) a polypeptide consisting of an amino acid sequence having a sequence identity of at least 80% to the amino acid sequence of SEQ ID NO: 4, 6, 8, 10, 12, 14, 16 or 18, and the polypeptide having kinase activity; and
(c) an SLC12A2-NRG2 fusion polynucleotide encoding the polypeptide mentioned below:
(i) a polypeptide consisting of the amino acid sequence of SEQ ID NO: 20, 22, 24, 26 or 28,
(ii) a polypeptide consisting of an amino acid sequence derived from the amino acid sequence of SEQ ID NO: 20, 22, 24, 26 or 28 by deletion, substitution or addition of one or more amino acids, and the polypeptide having intracellular signaling-enhancing activity, or
(iii) a polypeptide consisting of an amino acid sequence having a sequence identity of at least 80% to the amino acid sequence of SEQ ID NO: 20, 22, 24, 26 or 28, and the polypeptide having intracellular signaling-enhancing activity.

3. The method according to claim 1, wherein the fusion polynucleotide is any one of (a) to (c) below:
(a) (i) a polynucleotide consisting of the nucleotide sequence of SEQ ID NO: 1,
(ii) a polynucleotide that hybridizes under stringent conditions with a polynucleotide consisting of a nucleotide sequence complementary to the polynucleotide consisting of the nucleotide sequence of SEQ ID NO: 1, and which encodes a polypeptide having kinase activity,
(iii) a polynucleotide that consists of a nucleotide sequence derived from the nucleotide sequence of SEQ ID NO: 1 by deletion, substitution or addition of one or more nucleotides, and which encodes a polypeptide having kinase activity, or
(iv) a polynucleotide that has a sequence identity of at least 80% to the polynucleotide consisting of the nucleotide sequence of SEQ ID NO: 1, and which encodes a polypeptide having kinase activity;
(b) (i) a polynucleotide consisting of the nucleotide sequence of SEQ ID NO: 3, 5, 7, 9, 11, 13, 15 or 17,
(ii) a polynucleotide that hybridizes under stringent conditions with a polynucleotide consisting of a nucleotide sequence complementary to the polynucleotide consisting of the nucleotide sequence of SEQ ID NO: 3, 5, 7, 9, 11, 13, 15 or 17, and which encodes a polypeptide having kinase activity,
(iii) a polynucleotide that consists of a nucleotide sequence derived from the nucleotide sequence of SEQ ID NO: 3, 5, 7, 9, 11, 13, 15 or 17 by deletion, substitution or addition of one or more nucleotides, and which encodes a polypeptide having kinase activity, or
(iv) a polynucleotide that has a sequence identity of at least 80% to the polynucleotide consisting of the nucleotide sequence of SEQ ID NO: 3, 5, 7, 9, 11, 13, 15 or 17, and which encodes a polypeptide having kinase activity; and
(c) (i) a polynucleotide consisting of the nucleotide sequence of SEQ ID NO: 19, 21, 23, 25 or 27,
(ii) a polynucleotide that hybridizes under stringent conditions with a polynucleotide consisting of a nucleotide sequence complementary to the polynucleotide consisting of the nucleotide sequence of SEQ ID NO: 19, 21, 23, 25 or 27, and which encodes a polypeptide having intracellular signaling-enhancing activity,
(iii) a polynucleotide that consists of a nucleotide sequence derived from the nucleotide sequence of SEQ ID NO: 19, 21, 23, 25 or 27 by deletion, substitution or addition of one or more nucleotides, and which encodes a polypeptide having intracellular signaling-enhancing activity, or
(iv) a polynucleotide that has a sequence identity of at least 80% to the polynucleotide consisting of the nucleotide sequence of SEQ ID NO: 19, 21, 23, 25 or 27, and which encodes a polypeptide having intracellular signaling-enhancing activity.

4. The method according to claim 1, wherein the cancer is gastric cancer.

5. A method for identifying a patient with cancer or a subject with a risk of cancer, in whom a substance suppressing the expression and/or activity of a polypeptide encoded by a fusion polynucleotide produced by a gene fusion serving as a responsible mutation (driver mutation) for cancer shows a therapeutic effect, the method comprising the steps of:
(1) detecting a fusion polynucleotide of any one of (a) to (c) below, or a polypeptide encoded thereby, in an isolated sample from the subject:
(a) an ATG3-EPHB1 fusion polynucleotide which encodes a polypeptide comprising the kinase domain of EPHB1 and having kinase activity,
(b) a TNIK-RNF123 fusion polynucleotide which encodes a polypeptide comprising the kinase domain of TNIK and the SPRY domain and RING finger domain of RNF123, and having kinase activity, and
(c) an SLC2A2-NRG2 fusion polynucleotide which encodes a polypeptide comprising the AA_permease_N super family domain of SLC12A2 and the neuregulin family conserved region of NRG2, and having intracellular signaling-enhancing activity; and
(2) determining that the substance suppressing the expression and/or activity of the polypeptide shows a therapeutic effect in the subject, in the case where the fusion polynucleotide of any one of (a) to (c) or the polypeptide encoded thereby is detected.

6. A kit for detecting a gene fusion serving as a responsible mutation (driver mutation) for cancer, the kit comprising any or a combination of (A) to (C) below:
(A) a polynucleotide that serves as a probe designed to specifically recognize an ATG3-EPHB1 fusion polynucleotide, a TNIK-RNF123 fusion polynucleotide, or an SLC12A2-NRG2 fusion polynucleotide;
(B) polynucleotides that serve as a pair of primers designed to enable specific amplification of an ATG3-EPHB1 fusion polynucleotide, a TNIK-RNF123 fusion polynucleotide, or an SLC12A2-NRG2 fusion polynucleotide; and
(C) an antibody that specifically recognizes an ATG3-EPHB1 fusion polypeptide, a TNIK-RNF123 fusion polypeptide, or an SLC12A2-NRG2 fusion polypeptide.

7. An isolated ATG3-EPHB1 fusion polypeptide or a fragment thereof, which comprises the kinase domain of EPHB1 and has kinase activity.

8. An isolated TNIK-RNF123 fusion polypeptide or a fragment thereof, which comprises the kinase domain of TNIK and the SPRY domain and RING finger domain of RNF123, and has kinase activity.

9. An isolated SLC12A2-NRG2 fusion polypeptide or a fragment thereof, which comprises the AA_permease_N super family domain of SLC12A2 and the neuregulin family conserved region of NRG2, and has intracellular signaling-enhancing activity.

10. A polynucleotide encoding the fusion polypeptide or the fragment thereof according to claim 7.

11. A method for treating cancer positive for ATG3-EPHB1, TNIK-RNF123 or SLC12A2-NRG2 gene fusion, comprising administering, to a patient in need thereof, an effective amount of a substance suppressing the expression and/or activity of: an ATG3-EPHB1 fusion polypeptide comprising the kinase domain of EPHB1 and having kinase activity; a TNIK-RNF123 fusion polypeptide comprising the kinase domain of TNIK and the SPRY domain and RING finger domain of RNF123 and having kinase activity: or an SLC12A2-NRG2 fusion polypeptide comprising the AA_permease_N super family domain of SLC12A2 and the neuregulin family conserved region of NRG2, and having intracellular signaling-enhancing activity.

12. (canceled)

13. The method according to claim 11, wherein the cancer is gastric cancer.

14. A method for screening a cancer therapeutic agent, the method comprising the steps of:
(1) bringing a test substance into contact with a cell that expresses: an ATG3-EPHB1 fusion polypeptide comprising the kinase domain of EPHB1 and having kinase activity; a TNIK-RNF 123 fusion polypeptide comprising the kinase domain of TNIK and the SPRY domain and RING finger domain of RNF123 and having kinase activity; or an SLC12A2-NRG2 fusion polypeptide comprising the AA_permease_N super family domain of SLC12A2 and the neuregulin family conserved region of NRG2, and having intracellular signaling-enhancing activity;
(2) determining whether the expression and/or activity of any of said fusion polypeptides is suppressed or not; and
(3) selecting the substance determined to suppress the expression and/or activity of any of said fusion polypeptides, as a cancer therapeutic agent.

15. A polynucleotide encoding the fusion polypeptide or the fragment thereof according to claim 8.

16. A polynucleotide encoding the fusion polypeptide or the fragment thereof according to claim 9.
